(19) United States
(12) Patent Application Publication
(10) Pub. No.: US 2015/0086976 A1
(43) Pub. Date: Mar. 26, 2015

(54) SELF-CATALYTIC REACTION BASED ASSAY

(71) Applicant: Liaohai Chen, Logan, UT (US)

(72) Inventor: Liaohai Chen, Logan, UT (US)

(73) Assignee: UTAH STATE UNIVERSITY, North Logan, UT (US)

(21) Appl. No.: 14/493,290

(22) Filed: Sep. 22, 2014

Related U.S. Application Data
(60) Provisional application No. 61/880,573, filed on Sep. 20, 2013.

(51) Int. Cl. G01N 33/543; G01N 21/80; G01N 33/552

(52) U.S. Cl. CPC G01N 33/54306; G01N 33/552; G01N 21/80
USPC 435/5; 435/7.1; 436/501; 435/7.23

(57) ABSTRACT

A method for determining the presence or concentration of an analyte in a sample is described, including the steps of associating an initiator with the analyte to form an analyte associated initiator, contacting the analyte associated initiator with a reagent adapted to undergo a self-catalytic reaction which produces a product, and obtaining an observation or measurement corresponding to a change in the amount of the product. Kits and additional methods related to analyte detection are also described.

### CROSS-REFERENCE TO RELATED APPLICATIONS

This application claims priority to U.S. Provisional Patent Application No. 61/880,573, filed Sep. 20, 2013, which is incorporated herein in its entirety by reference.

### SEQUENCE LISTING

The sequence listing is filed with the application in electronic format only and is incorporated by reference herein. The sequence listing text file “ASFILED_SequenceListing-Text”, as document number: 15677784, was created on Sep. 22, 2014, and is 2839 bytes in size.

### FIELD OF INVENTION

The disclosure provided herein relates to bioassays that incorporate a self-catalytic reaction and methods and kits incorporating the same.

### BACKGROUND

A rapid and portable bioassay requiring minimal use of sophisticated instruments and reagents, but that is nonetheless sensitive enough for early detection of diseases biomarkers, pathogenic targets, and other analytes, is a grand challenge for the fields of biomedical diagnostics, counter terrorism, and disease monitoring and prevention. Although many of the commonly used approaches for detecting an analyte, including enzyme linked immunosorbant assays (ELISAs) and polymerase chain reaction (PCR), have high sensitivity and specificity, they require advanced instruments or multiple reagents, and are thus may not be suitable in resource limited settings such as third world countries, routine clinical use, home use, field use or use by first responders, where results are expected in minutes instead of hours or days.

An ELISA is an assay that uses antibodies and color changes to identify a substance or an analyte. It is a popular format of a “wet-lab” type analytic biochemistry assay that uses a solid-phase enzyme immunoassay (EIA) to detect the presence of a substance, usually an antigen, in a liquid or wet sample. ELISAs have been used as diagnostic tools in medicine and plant pathology, as well as for quality-control checks in various industries.

PCR is a biochemical technology used in molecular biology to amplify a single or a few copies of a piece of DNA across several orders of magnitude, generating thousands to millions of copies of a particular DNA sequence.

Various colorimetric sensors have been developed, including the use of chemically responsive dyes, polyacrylonitrile membranes, polydiacetylene (PDA), and graphene stacked films. While most of these sensors were designed to detect volatile organic compounds, the unique surface plasmon properties of gold nano-particles at different agglomeration states have been harnessed to construct different types of biological assays or sensors ranging from pregnancy test kits to the detection of DNA, pathogens, or enzymes. However, several drawbacks have limited the application of gold nano-particle-based colorimetric assays in real samples due to their instability in biological fluids, their target-specific design for an individual target, and their relatively low sensitivity and narrow dynamic range. A novel, quick, more sensitive, and more generic assay is needed for use in resource-limited environments where the assay must give the results quickly in order to enable medical personnel to make decisions on site, as patients may not be able to afford follow-up clinical visits.

An important principle of any detection assay/sensor lies in coupling the presence of the target with a transducer that converts the presence of the target or its biochemical activities to the measurement of a signal. Generally, the signal needs to be read or measured by an instrument such as a spectrophotometer or radioactivity counter. If the signal produces a dramatic change in color in the visible portion of the light spectrum, however, then the signal can be read by the human eye. A colorimetric visualization assay in which the signal is interpreted by the human eye offers an ideal disease diagnostic modality in a resource-limited environment, as it requires no additional instrumentation.

Herein, methods for detecting an analyte are disclosed, which incorporate a self-catalytic reaction initiated by a target associated initiator, the product of which may be visualized to determine the signal. Under certain conditions, the signal may be read by the human eye.

### SUMMARY

The present invention relates to methods for determining the presence or concentration of an analyte in a sample, comprising the steps of associating an initiator with the analyte to form an analyte associated initiator, contacting the analyte associated initiator with a reagent adapted to undergo a self-catalytic reaction when contacted with the initiator, wherein the self-catalytic reaction produces a product, and obtaining an observation or measurement corresponding to a change in the amount of the product.

The present invention also provides for a kit for detecting an analyte, comprising an initiator, an associating agent adapted to associate the initiator with the analyte to form an analyte associated initiator, a reagent adapted to undergo a self-catalytic reaction when contacted with the initiator, and instructions for their use.

In addition, the present invention relates to a kit for detecting cancerous or abnormal mammalian cells that have an enlarged cell nucleus when compared to normal cells, comprising hemalum, tert-butyl 2-methyl-2-(tosyloxymethyl) acetoacetate, and instructions for their use.

### BRIEF DESCRIPTION OF THE DRAWINGS

The drawings below are supplied in order to facilitate understanding of the Description and Examples provided herein.

FIG. 1 is a schematic depicting an exemplary acid initiated self-catalysis reaction. Tert-butyl 2-methyl-2-(tosyloxymethyl) acetoacetate, abbreviated as BMTA (Compound 1), is dissolved in an organic solvent. When in contact with a trace amount of initiator, which may be introduced to the system by the presence of disease biomarkers or pathogenic targets, it will dissociate into 2-methyl-3-oxobutyl 4-methylbenzenesulfonate (Compound 2). Compound 2 is unstable and generates p-toluene sulfonic acid (TsOH), which will reactivate the dissociation of BMTA.

FIG. 2 is a schematic showing a general scheme for an exemplary target analyte specific detection platform, incorporating a particle loaded with an initiator for the self-catalytic reaction.

FIG. 3 is a graph of optical density at 524 nm vs. the concentration of an exemplary acid-loaded silica particle.

FIG. 4A is a schematic showing an exemplary analyte specific detection platform.

FIG. 4B is a schematic showing an exemplary analyte specific detection platform, where the analyte is a nucleic acid.

FIG. 4C is a schematic showing an exemplary analyte specific detection platform for the analysis of a series of oligonucleotides.

FIG. 4D is a schematic showing an exemplary analyte specific detection platform, where the analyte is an antigen.

FIG. 4E is a schematic showing an exemplary analyte specific detection platform, where the analyte is an antigen.

FIG. 5 is a schematic showing an exemplary analyte specific detection platform, where the target analyte is an abnormal or cancerous cell having a larger nucleus than that of a normal cell.

FIG. 6 is a graph of the relative optical density at 550 nm of a self-catalytic reaction solution as a function of the amount of hemalum added (in log scale), in an exemplary assay.

FIG. 7A is a graph showing the absorption at 524 nm of self-catalytic reaction solutions over a total of 45 exemplary assays.

FIG. 7B is a bar chart showing the absorbance of the indicator solutions of FIG. 7A plotted for both HeLa and End1 cells.

FIG. 8 is a graph showing the optical density at 524 nm of a self-catalytic reaction solution as a function of the amount of hemalum added, in an exemplary assay.

FIG. 9 is a bar chart showing the amount of hemalum associated with a specific number of HeLa and End1 cells as determined by HPLC, in an exemplary assay.

FIG. 10 is a schematic showing a bioassay for detecting a target DNA according to embodiments of the present disclosure.

### DETAILED DESCRIPTION

This disclosure is not limited in its application to the details of construction and the arrangement of components set forth in the following description. The disclosure may provide other embodiments and may be practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also should be understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

It should be understood that, as used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

Reference throughout this specification to “one embodiment,” “an embodiment,” or similar language means that a particular feature, structure, or characteristic described in connection with the embodiment is included in at least one embodiment of the invention provided herein. Thus, appearances of the phrases “in one embodiment,” “in an embodiment,” and similar language throughout this specification may, but do not necessarily, all refer to the same embodiment.

Furthermore, the described features, structures, or characteristics of the methods, compositions, and kits provided herein may be combined in any suitable manner in one or more embodiments. In the following description, numerous specific details are provided, to provide a thorough understanding of embodiments. One skilled in the relevant art will recognize, however, that the embodiments may be practiced without one or more of the specific details, or with other methods, components, materials, and so forth. In other instances, well-known structures, materials, or operations are not shown or described in detail to avoid obscuring aspects of the embodiments.

The depicted order and labeled steps depicted in schematic diagrams are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present disclosure provides methods and kits for determining the presence or concentration of an analyte in a sample. Generally, methods may include associating an initiator with the analyte to form an analyte associated initiator, contacting the analyte associated initiator with a reagent adapted to undergo a self-catalytic reaction when contacted with the initiator, wherein the self-catalytic reaction produces a product, and obtaining an observation or measurement corresponding to a change in the amount of the product.

Self-Catalytic Reactions.

A self-catalytic reaction is a chemical or biological reaction in which at least one reactant is also a product of the reaction. An example of a self-catalytic reaction may include a reagent that reacts with an initiator to form a product, where the product then reacts with more reagent to form more product. In other words, some self-catalytic reactions may be initiated by the initiator. In some embodiments, the product, or a change in the amount of the product, may be detectable. The product may be directly measured, or may be indirectly measured, such as when a product subsequently reacts with one or more other compounds in a manner that is observable or detectable. For example, the product may be a colorant that may be visualized as the product is formed. Another exemplary product of the self-catalytic reaction may include a proton, which is the product of an acid producing or acid amplifying self-catalytic reaction. The protons produced by an acid amplifying self-catalytic reaction may be observed or measured, e.g., with a pH dependent indicator, such as an indicator dye. As will be appreciated by those skilled in the art, an observation and/or a measurement corresponding to the formation of a product, or a change in the amount of a product, may be visualized by the human eye, or may be detected by analytical instrumentation, such as a spectrophotometer.

Self-catalytic reactions thus may be used as means for detecting the presence or concentration of the initiator. By that same virtue, as described in more detail below, if the initiator is associated with some other analyte (e.g., to form an analyte-associated initiator), the reaction may be used to detect the presence or concentration of the analyte. Moreover, because the reaction is self-catalytic, a substantially greater amount of product may be generated relative to the amount of initiator or analyte, and as such, the self-catalytic reaction may be used to amplify a signal corresponding to the amount of initiator or analyte in a sample.

Target Analytes.

Analytes may include any biological or chemical target analyte including, but not limited to, cells, viruses, proteins, hormones, antibodies, antigens, receptors, ligands, oligonucleotides, peptides, or any other chemical or biological compound or composition.

Reagents.

Examples of reagents that undergo self-catalytic reactions to form detectable or observable products may include, but are not limited to, acid proliferation reactions, base proliferation reactions, templated self-replication reactions, nitrobenzene oxidation by hydrogen peroxide and Fe(III), and pinacol fragmentation with carbon tetrachloride. The reagent may form products in a linear or a non-linear manner. In some embodiments, the reagent may comprise an acid amplifier, which refers to a reagent that is initiated by an acid and which also produces acid as a product during the reaction. Examples of acid amplifiers may include, but are not limited to, tert-butyl 2-methyl-2-(tosyloxymethyl) acetoacetate, pinanediol monotosylate, a benzyl sulfonate, an acetoacetate derivative, an alpha-ketal sulfonate, a 1,2-diol monosulfonate, a trioxane derivative, a cyclohexane-1,4-disulfonate, and a polymer-bound pinanediol monosulfonate.

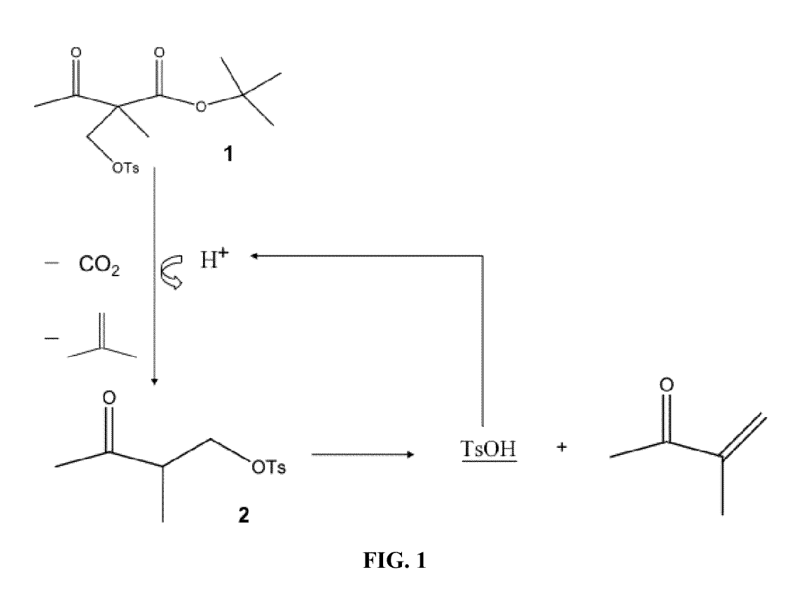

For example, FIG. 1 shows the self-catalytic reaction of the acid amplifier tert-butyl 2-methyl-2-(tosyloxymethyl) acetoacetate (BMTA) In the presence of the initiator, the reaction of yields the intermediate product 2-methyl-3-oxobutyl 4-methylbenzenesulfonate. In the presence of an acid initiator, BMTA undergoes a spontaneous decomposition reaction to produce p-toluene sulfonic acid, which can further catalyze the reaction of BMTA until all the BMTA is transformed to p-toluene sulfonic acid. After the self-catalytic reaction cycles, the formation of the large amount of p-toluene sulfonic acid produced can be visualized by a pH-indicating dye. If BMTA is introduced into a system that comprises an analyte that has been associated with an acid initiator, the BMTA will react with the initiator, thus initiating the self-catalytic reaction, and transforming the BMTA to p-toluene-sulfonic acid. A measurable or detectable change in the pH caused by this reaction may thereby indicate the presence of the analyte.

Initiators.

Initiators may include any chemical or biological molecule capable of initiating a self-catalytic reaction to form a product that further catalyzes the self-catalytic reaction. Initiators may be acids, photolytic initiators, thermal initiators, cellular components that are constituents of a cell, diseased cell, or treated cell, or any combination of the foregoing. For example, a disease biomarker or pathogen that is acidic may initiate an acid amplifying self-catalytic reaction, and in some cases may also act as the target analyte. Initiators may be associated with a target analyte to form an analyte-associated initiator (also described herein as a target-associated initiator), which include target analytes acting as initiators, initiators that are directly physically or chemically associated with a target analyte, and initiators that are physically or chemically associated with an analyte through the use of an associating agent.

Acidic initiators may include both organic or inorganic acids, and may be hydrophobic or hydrophilic, depending on the application. Examples of hydrophobic organic acids may include, but are not limited to, dodecyl p-toluenesulfonate, ethylene di(p-toluenesulfonate), 2-butynyl p-toluenesulfonate, phenyl p-toluenesulfonate, tetrasodium pyrophosphate, or p-toluenesulfonic acid. Another exemplary acid initiator may include a cell staining agent, such as hemalum, which is known to color the nucleus of cells.

Analyte-Associated Initiators.

If the target analyte itself cannot function as an initiator for the self-catalytic reaction, then an initiator may be physically or chemically associated or coupled with the target analyte. In some embodiments, the initiator may be directly and specifically bound to or otherwise associated with the target analyte. For example, if the target analyte is a cell having an enlarged cell nucleus (e.g., a cancer cell or some other abnormal cell having an enlarged nucleus), then the cell may be stained with an acidic staining molecule, such as a dye-metal complex, including, for example, hemalum, an iron-hematein complex, brazalum and carmalum. In some embodiments, the initiator may be associated with the target analyte by coupling the initiator with an associating agent, which may be adapted to specifically interact with the analyte, thereby associating the initiator with the analyte to form an analyte-associated initiator.

Associating Agents.

As indicated above, some initiators may be associated with a target analyte using an associating agent to form an analyte-associated initiator. Exemplary associating agents may include a binding moiety adapted to specifically bind to the analyte. Binding moieties may include, but are not limited to, antibodies, antigens, polynucleotides, proteins, or small molecules, or any other suitable ligand. As will be appreciated by those skilled in the art, binding moieties may rely on antibody-antigen interactions, protein-protein interactions (e.g., biotin-avidin), the complimentary binding of nucleic acids, or ligand-receptor interactions including small molecule ligands and synthetic receptors. For example, a binding moiety may include folic acid or a derivative thereof, which may bind specifically to a folate receptor. Some mammalian cancer cells overexpress the folate receptor, and as such, an associating agent having a folic acid binding moiety may be used to associate an initiator to a mammalian cell that overexpresses the folate receptor.

In some embodiments, the binding moiety may be coupled (e.g, directly or indirectly attached) to the initiator. In other embodiments, the binding moiety may be coupled (e.g, directly or indirectly attached) to a carrier, which carries the initiator in a manner that associates the initiator with the target analyte when the binding moiety binds to the target analyte. In other words, the carrier may be linked or otherwise coupled to the target analyte, thus bringing the initiator in close proximity to the target analyte. The initiator may be carried by the carrier in a variety of ways, such as by being absorbed, adsorbed, bound, chelated or infused onto the surface of the carrier, or into an interior space of the carrier. For example, the carrier may be a porous particle (e.g., a porous silica particle), and the initiator may be infused into the pores of the particle. The carrier and the initiator may be selected so that the carrier releases the initiator (or the initiator diffuses away from the carrier) at a relatively slow rate. This may allow sufficient time to associate the initiator with the target analyte, and then contact a reagent of a self-catalyzing reaction with the analyte-associated initiator so that the initiator and the reagent react with one another to catalyze the self-catalyzing reaction. In some embodiments, the carrier may be adapted to release the initiator at a slow enough rate such that the carrier continues to carry sufficient quantities of the initiator despite putting the associating agent and/or a complex formed from the associating agent and the analyte through one or more purification steps.

Methods for Determining the Presence of Concentration of an Analyte in a Sample.

As indicated above, the methods of this disclosure may include associating an initiator with the analyte to form an analyte associated initiator, contacting the analyte associated initiator with a reagent adapted to undergo a self-catalytic reaction when contacted with the initiator, wherein the self-catalytic reaction produces a product, and obtaining an observation or measurement corresponding to a change in the amount of the product.

In some embodiments, it may be desirable to specifically bind the analyte to a solid support so as to be able to separate the analyte from the rest of the compounds and compositions in a sample, such as by washing the solid support. For example, the solid support may be conjugated with a binding moiety adapted to specifically bind to the analyte, and then the solid support may be washed to remove proteins and other molecules that do not specifically bind to the solid support. This wash step may be performed before and/or after associating the initiator with the analyte. Washing the solid support after associating the initiator with the analyte further may remove any initiator that is not specifically associated with the analyte. It should be appreciated that a binding moiety attached to the solid support may be the same or different than a binding moiety of an associating agent. In either case, the binding moieties of the solid support and the associating agent may “sandwich” the target analyte, similar to an ELISA assay. As such, the initiator may be coupled to the solid support only in the presence of the analyte (e.g., via a carrier, a binding moiety coupled to the carrier of the associating agent, the analyte, and the binding moiety of the solid support). A starting reagent of a self-catalytic reaction may then be applied to the solid support, whereupon the initiator may initiate the self-catalytic reaction to produce detectable reaction products.

A solid support refers to a solid support material from which at least one of the components of the bioassay is attached. The solid support may be an immunoassay plate, an uncoated or coated glass slide, or a particle. The solid support may be magnetic or non-magnetic, or it may be radioactive or non-radioactive. The solid support may be used to attach any one of, or multiple components of the bioassay, including the initiator, a binding moiety, a linking moiety or an analyte.

In some embodiments, in order to determine the presence or absence of a target analyte in an unknown sample, it may be necessary to analyze both the unknown sample and one or more control samples having known concentrations of analyte, and comparing observations or measurements associated with each sample to determine the presence or concentration of the analyte in the unknown sample. More specifically, to the extent the methods disclosed herein comprise the step of obtaining an observation or measurement corresponding to a change in the amount of the product produced from the self-catalytic reaction initiated using an unknown sample, the method further may comprise comparing the observation or measurement associated with the unknown sample to an analogous observation or measurement associated with at least one control sample, and determining the presence of concentration of the analyte in the sample based on the comparison. For example, when using the bioassay to screen for cancerous cells, it may be useful to incorporate the screening of a known abnormal cell in addition to a known non-cancerous cell, as control or standardized samples to determine the type of response, such as the exact color or color intensity of a pH-dependent indicatory, which constitutes a positive and negative response.

The methods disclosed herein may be used to identify the presence or concentrations of may analytes, as detailed above. For example, the methods may be used to identify the presence of a genetic mutation in a portion of nucleic acid, in which comparison of the observation or measurement associated with a known standard portion of nucleic acid (i.e. the response of the assay to the control sample) to the observation or measurement associated with the unknown sample (i.e. the response of the assay to an unknown sample) will determine if the mutation is present in the unknown sample, based upon the presence or concentration of the analyte.

The methods also may be used to identify certain cancers. Cervical cancer is the most common cancer among women in the developing world. In Africa, more than half a million women get cervical cancer and more that 275,000 die from it each year. Similar rates are also observed in some parts of Latin America. However, cervical cancer is treatable when diagnosed early. Unfortunately, fewer than five percent of women in developing countries have access to screening even once in their lifetime. Many tests currently used in the developed world cannot be applied due to the impracticality of the technology and/or limited resources of the developing country. To fill this gap, a self-catalytic colorimetric visualization (SCCV) assay using the methods disclosed herein, may be used for detecting cervical cancer cells with visualization by the human eye.

In a low-resource setting, an ideal disease diagnostic modality is a bioassay that can be performed in minutes by untrained personnel. Herein is disclosed the development of a self-catalytic colorimetric assay to detect the existence of cervical cancer cells by the human eye. The assay comprises a target specific initiator, an acid self-catalytic system, and a pH indicator solution. By taking advantage of the presence of a trace amount of the nucleus staining molecule, hemalum, which can function as the initiator, a stained cervical cancer cell with an enlarged nucleus can initiate the self-catalytic reaction under a defined reaction condition to produce a significant amount of acid, which leads to a color change of a pH indicator solution, thereby indicating the presence of the cancerous cell. Amplification of the number of protons by a factor of at least about a trillion-fold as compared to the number of protons which initiate the self-catalytic reaction, may be achieved within 1.5 minutes.

Due to the minimal use of instrumentation, its simplicity and low cost, the bioassay is a potential tool for clinicians and may function as a “yes/no” check to facilitate quick decisions for cervical cancer screening. Furthermore, by alternating the processes whereby the initiator is associated with the target, the assay can be expanded to screen and detect protein or nucleic acid based biomarkers related to other epidemic diseases in resource-limited settings.

The methods disclosed herein also may be applied to determining the presence of mutated genomic DNA, which may be useful in a variety of circumstances, including the detection of specific viruses or diseases. One exemplary disease is sickle cell disease (SCD). By using oligonucleotides complementary to a portion of genomic DNA related to SCD as capture and affinity reagents (capture and reporter oligos thereafter), the presence of mutated genomic DNA via a simple colorimetric visualization may be achieved. Such methods take advantage of the accuracy of DNA analysis by directly detecting the presence of mutated genetic materials related to SCD without involvement of any analytic instrumentation. More importantly, the assay can be performed without specific training, which makes the method an ideal detection modality for SCD screening in a resource-limited setting. Potential markets also include home based screening kits for enhanced privacy.

Visualization of Results.

Visualization of a product of the self-catalytic reaction may be achieved by a variety of means. For example, visualization may be achieved using the human eye. In general, the human eye can easily distinguish a 5% change in the color of a solution. For a colorant with a relatively high extinction coefficient (for example, ε=50,000), such as the methyl red pH-dependent indicator, the number of new colorant molecules needed to induce a distinguishable color change in a fixed volume of solution (using a 1 cm path length and 1 ml volume) can be calculated based on the Beer-Lambert law {Log(100/95)=50,000×1×[colorant]. Alternatively, visualization may be achieved with a spectrometer that is capable of differentiating color based on absorption. In yet another example, visualization may be achieved with the use of a camera. Pictures from the camera may be processed on a computer. In addition to visualizing a color change, visualization also includes observing the change of color patterns.

In various embodiments, the present invention relates to methods for determining the presence or concentration of an analyte in a sample, comprising associating an initiator with the analyte to form an analyte associated initiator, contacting the analyte associated initiator with a reagent adapted to undergo a self-catalytic reaction when contacted with the initiator, wherein the self-catalytic reaction produces a product, and obtaining an observation or measurement corresponding to a change in the amount of the product.

Kits for Detecting an Analyte.

This disclosure also provides kits for detecting an analyte, comprising an initiator, an associating agent adapted to associate the initiator with the analyte to form an analyte associated initiator, a reagent adapted to undergo a self-catalytic reaction when contacted with the initiator, and instructions for their use. The various components of the kit are described in more detail above with reference to the methods.

### EXAMPLES

Exemplary embodiments of the present disclosure are provided in the following examples. The examples are presented to illustrate the inventions disclosed herein and to assist one of ordinary skill in making and using the same. The examples and not intended in any way to otherwise limit the scope of the inventions disclosed herein.

### Example 1

### Exemplary Bioassays Based on a Target Analyte—Initiator Association Through an Affinity Reagent Chemically Conjugated with an Initiator

A ligand (affinity reagent)—receptor (target analyte) assay was developed based on the self-catalytic system described herein, and coupled with a pH indicator solution that yields a color change upon exposure to a low copy number of disease biomarkers or pathogenic target analytes. Briefly, a modified substrate was used to capture avidin protein molecules and cancer cells, followed by exposure of the substrate to a ligand-initiator complex solution. The introduction of the substrate loaded with the target analyte receptor-ligand-initiator complex leads to initiating the self-catalytic reaction. Subsequently, the self-catalytic reactions yielded enough acid (pH<2) to be visualized by a pH indictor solution. In an embodiment, the system has the ability to rapidly detect the presence of receptor protein molecules in the picomolar range, and of cancer cells at the single cell level.

General Procedures. Dry solvents (THF, dichloromethane) were purchased and used without any additional drying processing. All reagents were used as purchased without further purification unless otherwise stated. All reactions were performed under an atmosphere of nitrogen in oven-dried glassware. Routine monitoring of reactions was performed using silica gel TLC plates (Merck 60 F254). Spots were visualized by UV and/or dipping the TLC plate into a container of iodine vapor. Silica gel chromatography was carried out on silica gel 60 (200-400 mesh).

Physical measurements. ¹H and ¹³C NMR spectra were recorded at ambient temperature on a JEOL 300 and 75 MHz spectrometer, respectively. Chemical Shifts (δ) are reported in parts per million (ppm) referenced to CHCl₃ at 7.26 ppm for ¹H spectra, and CDCl₃ at 77.0 ppm for ¹³C spectra. Coupling constants are reported in Hertz (Hz), with the following abbreviations used: s=singlet, d=doublet, t=triplet, q=quartet, ABX=AB quartet, m=multiplet. When appropriate, the multiplicities are preceded with “br,” indicating that the signal was broad. High-resolution mass data were analyzed on an Agilent LCTOF (2006), a high resolution TOF mass spectrometer with APCI/ESI (multimode) capabilities. UV-Visible absorption of the indicator solutions was recorded on a Hewlett-Packard Model 8453 UV-Vis Spectrophotometer.

Synthesis. The acid amplifier tert-butyl 2-methyl-2-(p-toluenesulfonyloxymethyl) acetoacetate (BMTA) was synthesized by monomethylation of commercially available tert-butylacetoacetate at its active site. Aldol reaction of the methylated keto-ester resulted in hydroxymethylation, followed by tosylation of the hydroxyl group, to yield the desired auto acid precursor in a 28% overall yield.

The detailed synthetic procedure is as follows:

Tert-Butyl 2-methyl-3-oxobutanoate (1). To a suspension of sodium hydride (4.13 g 60% in mineral oil, 100 mmol) in anhydrous tetrahydrofuran (50 mL) was added tert-butylacetoacetate (15.82 g, 100 mmol) dropwise at 0° C. under nitrogen. The reaction was stirred for 1 h followed by the dropwise addition of a solution of methyl iodide (14.19 g, 100 mmol) in tetrahydrofuran under ice cooling, and the mixture was stirred 2 days at room temperature. After conventional workup and silica gel column chromatography using 10% ethyl acetate in hexane, a colorless oil was obtained (9.47 g, 55% yield). ¹H NMR (300 MHz, CDCl₃) δ 3.36 (q, J=7.1 Hz, 1H), 2.19 (s, 3H), 1.42 (s, 9H), 1.24 (d, J=7.2 Hz, 3H); ¹³C NMR (75.57 MHz, CDCl₃) δ 204.1, 169.9, 81.8, 54.7, 28.3, 27.9, 12.7; HRMS (ESI) calcd. for C₉H₁₇O₃ [M+H]⁺: 173.1178, found: 173.1179; calcd. for C₉H₂₀NO₃ [M+NH₄]⁺: 190.1443, found: 190.1446.

Tert-Butyl 2-(hydroxymethyl)-2-methyl-3-oxobutanoate (2). 20% Formaldehyde (16.5 g, 110 mmol, 16.5 mL) was added to a 1 M solution of tert-butyl 2-methyl-3-oxobutanoate (7.58 g, 44.0 mmol) in dioxane. The mixture was stirred at room temperature while a 1 M solution of triethylamine (2.2 mmol) in tetrahydrofurane was added. At this point, the temperature was raised to 35-40° C. and stirring was continued for 4 h. The reaction was coevaporated with dioxane (3×100 mL) and toluene (3×100 mL), dissolved in methylene chloride and separated on a silica gel column eluting with a gradient of ethyl acetate (0-30%) in methylene chloride to obtain a colorless oil (6.60 g, 74%). ¹H NMR (300 MHz, CDCl₃) δ 3.73 (ABX, JAB=11.3 Hz, JAX=7.2 Hz, JBX=6.5 Hz, 2H), 2.89 (dd, J=6.5 Hz, J=7.2 Hz, 1H), 2.14 (s, 3H), 1.39 (s, 9H), 1.26 (s, 3H); ¹³C NMR (75.57 MHz, CDCl₃) δ 206.8, 171.4, 82.5, 66.4, 61.8, 27.9, 27.0, 17.2; HRMS (ESI) calcd. for C₁₀H₁₈NaO₄ [M+Na]⁺: 225.1103, found: 225.1100; calcd. for C₁₀H₂₂NO₄ [M+NH₄]⁺: 220.1549, found: 220.1533.

Tert-Butyl 2-methyl-2-(p-toluenesulfonyloxymethyl)-3-oxobutanoate (3). Triethylamine (3.63 g, 5.00 mL) and 4-(dimethylamino)pyridine (1.33 g, 10.9 mmol) was added to a 1M solution of p-toluenesulfonyl chloride in methylene chloride. The mixture was stirred at room temperature for 15 minutes followed by the dropwise addition of a 1M solution of tert-butyl 2-(hydroxymethyl)-2-methyl-3-oxobutanoate in methylene chloride. The reaction was refluxed overnight, cooled to room temperature, diluted with ether, washed with saturated copper sulfate solution and brine respectively, finally dried over sodium sulfate to evaporate the solvent. The oily residue was purified by column chromatography on silica gel with a 10:1 mixture of hexane and ethyl acetate. Colorless crystals (7.85 g, 67%) were obtained by recrystallization from hexanes. ¹H NMR (300 MHz, CDCl₃) δ 7.77 (d, J=8.4 Hz, 2H), 7.35 (d, J=8.1 Hz, 2H), 4.27 (ABq, J=9.6, 2H), 2.44 (s, 3H), 2.11 (s, 3H), 1.41 (s, 9H), 1.37 (s, 3H); ¹³C NMR (75.57 MHz, CDCl₃) δ 203.0, 168.5, 145.2, 132.5, 130.0, 128.1, 83.2, 71.9, 60.2, 27.8, 26.1, 21.8, 17.4; HRMS (ESI) calcd. for C₁₇H₂₄NaSO₆ [M+Na]⁺: 379.1191, found: 379.1199; calcd. for C₁₇H₂₈NSO₆ [M+NH₄]⁺: 374.1637, found: 374.1637.

Affinity reagent conjugation. Organic acid loaded silica nanoparticles were synthesized via sol-gel process. 0.01 g of Tween 80 and 95 mg tosylic acid (TsOH) molecules were dissolved into 0.5 mL of 2M HCl aqueous solution, followed by the addition of 12.5 mL of cyclohexane solution containing 0.5 g of Span 80. After vigorous stirring for 20 min, the solution was probe-sonicated for a period of 2 min three times with 0.5 min intervals to form an emulsion solution. 0.45 mL of tetraethyl orthosilicate (TEOS) was added into the formed emulsion solution and stirred for 10 h at room temperature. For particle surface functionalization, 30 ul of (3-aminopropyl)triethoxysilane (APTS) or (3-mercaptopropyl) trimethoxy silane was added to the solution and the mixture was further incubated for 2 hr. The silica particles formed were isolated by washing with acetone, ethanol and water sequentially to remove any surfactant and free TsOH molecules. The particles were re-suspended in PBS buffer solution and the affinity reagent, such as biotin, folate, an antibody or an amine-modified oligonucleotide, was conjugated to the surface of the particle via standard amidation or via an amine-to-sulfhydryl conjugation using N-succinimidyl 3-(2-pyridyldithio)propionate as a cross-linking agent. For example, the conjugation of folate and biotin to the particle was achieved by using N,N′-dicyclohexyl carbodiimide (DCC) to link the folate carboxy group with the amine group on the surface of the particles.

Results

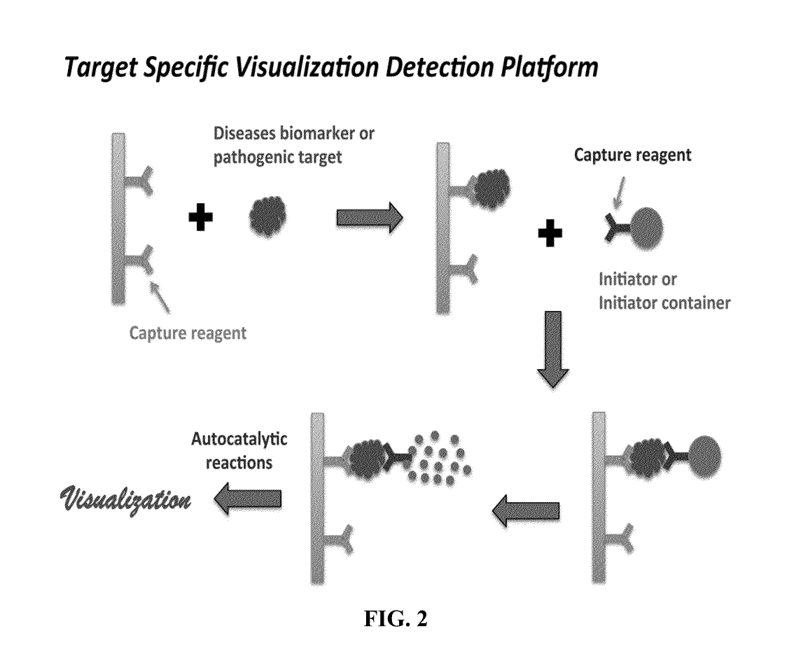

Sensor principle. As depicted in FIG. 1, the visualization assay consisted of a self-catalytic system. The self-catalytic reaction is initiated by a trace amount of acidic initiator. As shown in FIG. 2, a capture reagent modified substrate was used to capture the biomarker receptor molecules or pathogen target analytes. The initiator was chemically conjugated to a ligand molecule, which was specifically bound to the substrate due to the specific interactions between the ligand molecule and the biomarker receptor proteins or the corresponding receptors on the target cells. Once the substrate was introduced to the self-catalytic system, the initiator catalyzed the reaction of tert-butyl 2-methyl-2-(tosyloxymethyl) acetoacetate (BMTA) to yield the intermediate product of 2-methyl-3-oxobutyl 4-methylbenzenesulfonate. The intermediate product undergoes a spontaneous decomposition reaction to produce p-toluene sulfonic acid and the regeneration of original protons. Both p-toluene sulfonic acid and the original protons can further catalyze the reaction of BMTA until all the BMTA is transformed to p-toluene sulfonic acid. After the self-catalytic reaction cycles, the formation of the relatively large amount of p-toluene sulfonic acid can be visualized by a pH indicating dye, which in turn, indicates the presence of the biomarker receptor molecules or pathogen targets.

There are different ways to associate the initiator with the disease biomarkers or pathogenic target analytes. One approach involves the disease biomarker or pathogenic target analyte itself being the initiator by being acidic, thus the assay can detect the disease biomarker or pathogenic target analyte directly. The second approach is to associate the initiator molecule to the disease biomarker or pathogenic target analyte by utilizing a unique property of the disease biomarker or pathogenic target analyte that can harbor or associate the initiator directly. The third approach is to use a target analyte specific inhibitor (i.e. an affinity reagent—initiator complex/particle) to associate the initiator to the disease biomarker or pathogenic target analyte by ligand-receptor or antibody-antigen interactions (as shown in FIG. 2).

Silica nanoparticles were loaded with tosylic acid (TsOH) as the initiator for the self-catalytic reaction of BMTA. Subsequently, silica nanoparticles were labeled with biotin (against avidin as a model system) as well as folate as a cancer cell specific ligand on its surface, in which the TsOH initiator molecules were loaded inside the silica nanoparticles. Avidin is a tetrameric biotin-binding protein. It contains four identical subunits (homotetramer), each of which can bind to biotin (Vitamin B7, vitamin H) with a high degree of affinity and specificity. The dissociation constant of avidin is measured to be Kd≈10⁻¹⁵ M. It has been well established that folate receptors, which have a high affinity for folic acid, are overexpressed in a number of epithelial-derived tumors including ovarian, breast, renal, lung, colorectal, and brain. By labeling the silica nanoparticle with biotin or folate, ligand conjugated silica nanoparticles can specifically bind to avidin protein or cancer cells. Both SKOV3 and HeLa cancer cells were used in this study, while BSA protein and End1 normal epithelial cells were used as negative controls. Since the silica nanoparticles were loaded with initiator molecules, they triggered the self-catalytic reaction of BMTA, which subsequently can be conveniently visualized using a pH indicator.

Identifying a suitable initiator. More than 20 organic acids were screened to identify a suitable initiator for initiating the self-catalytic reaction, and they were selected based on their hydrophobicity, acidity and the efficiency of initiating the self-catalytic reaction. Hydrophobic acids were considered because their acidity is not sensitive to an aqueous environment before the hydrolysis in a biologic sensing system. The reaction can be easily followed by visualization after the addition of a pH indicator. Six acid candidates and the minimum amount of each acid needed to initiate the reaction in two different solvents is shown in the following table:

| Acid | In ACN (mg) | In CHCl₃ (mg) |
|---|---|---|
| DTS | 0.01-0.03 | 0.02-0.04 |
| ETS | 0.03 | >0.1 |
| BTS | 0.01 | 0.01 |
| PTS | >0.1 | >0.05 |
| TSPP | 0.001 | not tested |
| p-toluenesulfonic acid | 0.001 | not tested |

The six organic acids shown in the table are dodecyl p-toluenesulfonate (DTS), ethylene di(p-toluenesulfonate) (ETS), 2-butynyl p-toluenesulfonate (BTS), phenyl p-toluenesulfonate (PTS), tetrasodium pyrophosphate (TSPP), and p-toluenesulfonic acid (TsOH). Among the acids screened, p-toluenesulfonic acid had the highest sensitivity to initiate the reaction, as the smallest amount of TsOH was needed. Without wishing to be bound by theory, the hydrophobic acids are believed to be particularly useful because they don't dissociate from the silica particles during the washing steps. Rather than being dependent upon a specific pore size or type of specialized silica particle to provide appropriate association of an acid with the silica particles, the inventors surprisingly discovered that hydrophobic acids maintain their association with the silica particles when surrounded by an aqueous solution even when exposed to multiple and/or vigorous washing steps.

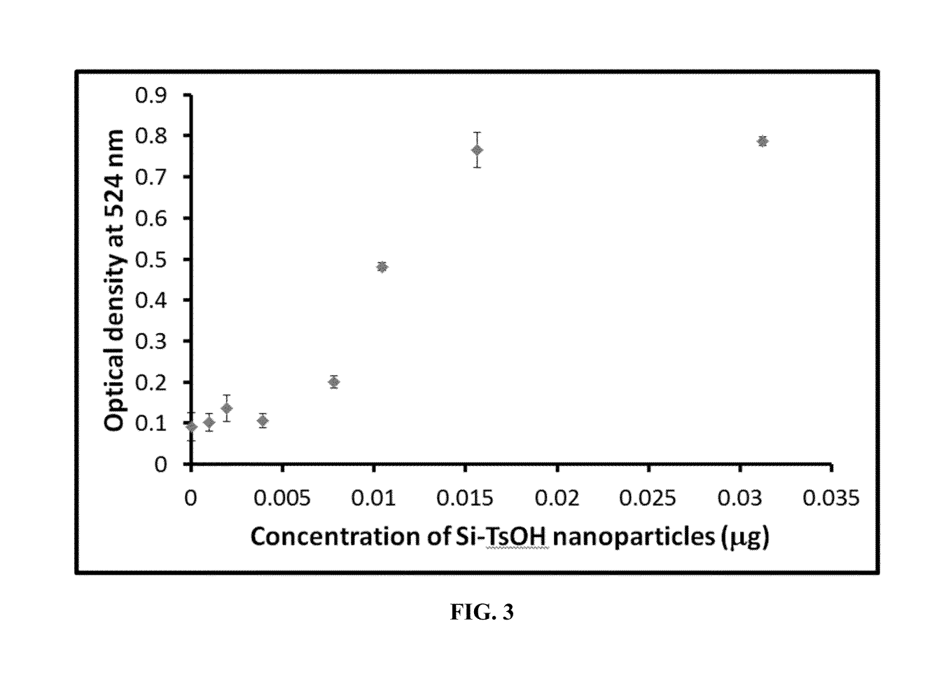

To facilitate the conjugation of a ligand with TsOH, as well as to enhance the assay sensitivity, silica nanoparticles were used as both the TsOH carrier and the scaffold for anchoring the ligand molecules. TsOH loaded silica nanoparticles were synthesized as described above. FIG. 3 shows that as little as about 5 nanograms of TsOH loaded silica nanoparticles can trigger the self-catalytic reaction effectively, when measured at 254 nm using methyl red (15 uM, freshly titrated with 0.1N NaOH) as an indicator.

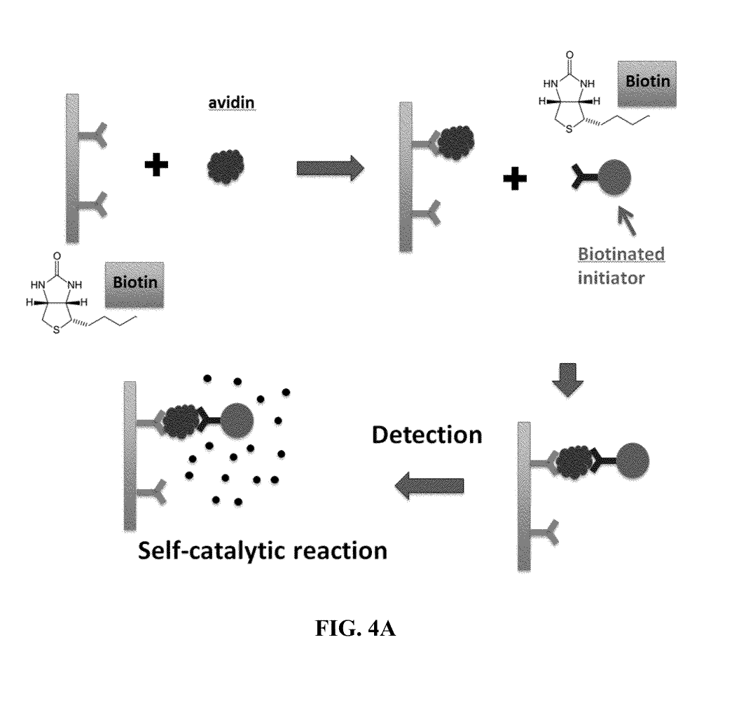

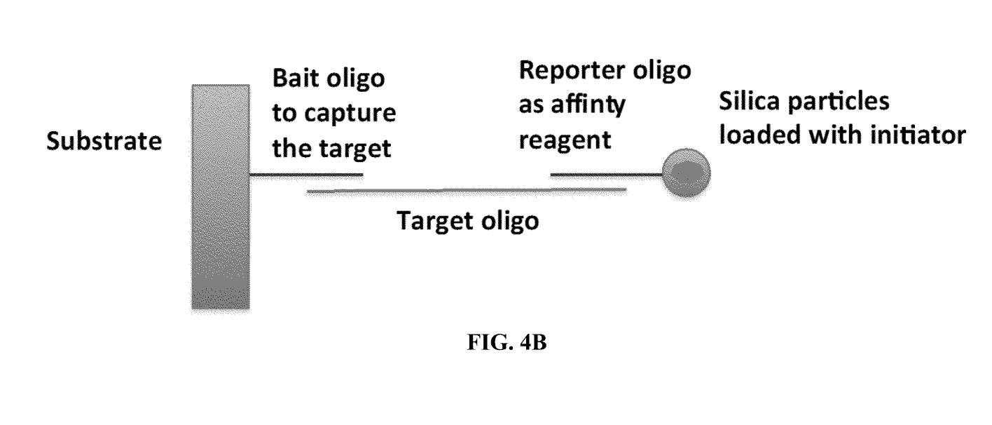

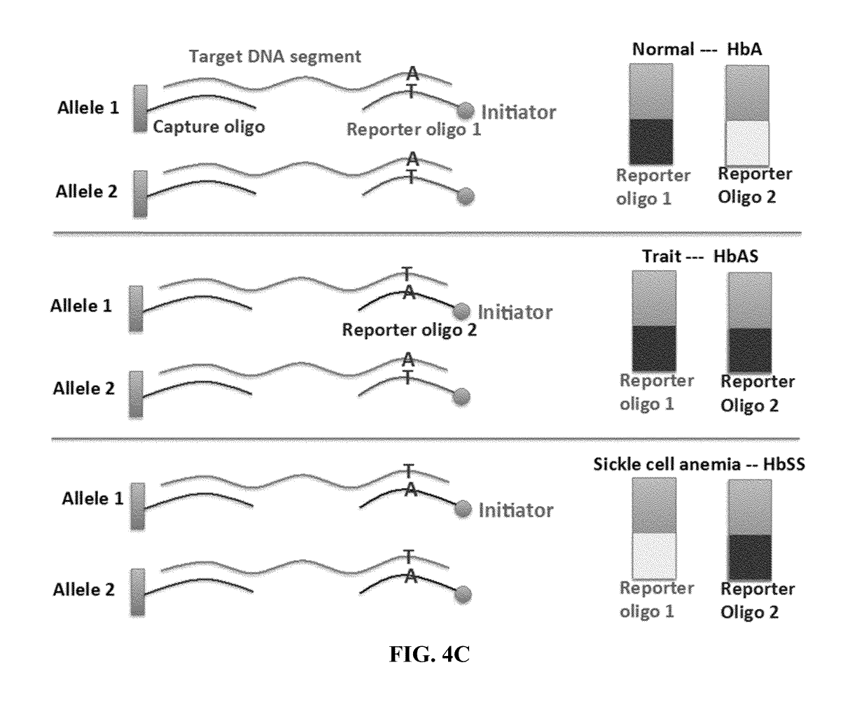

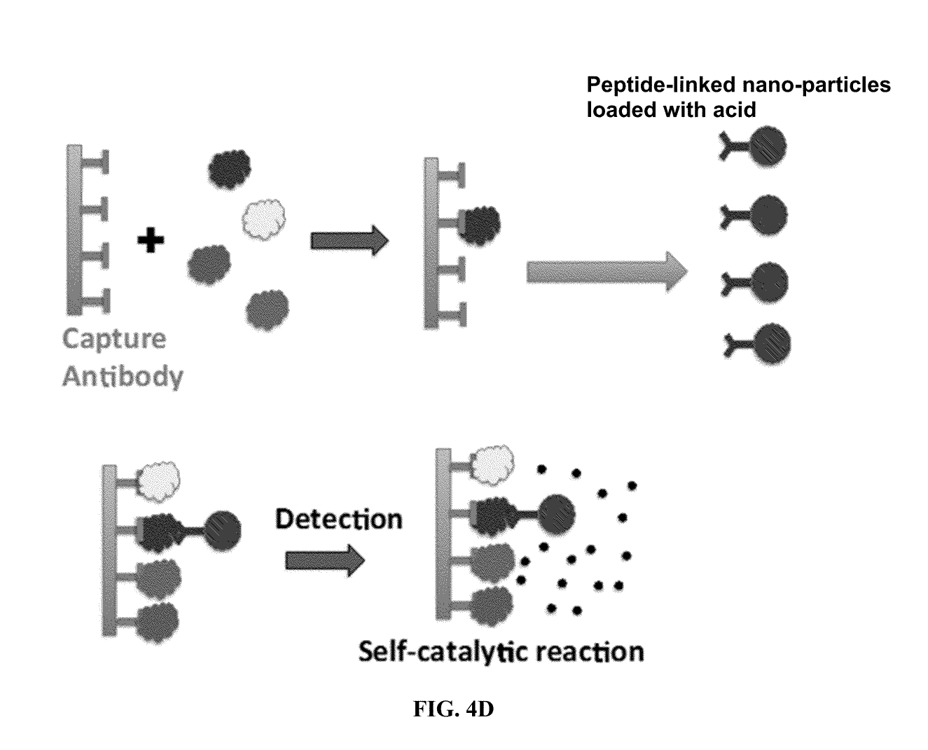

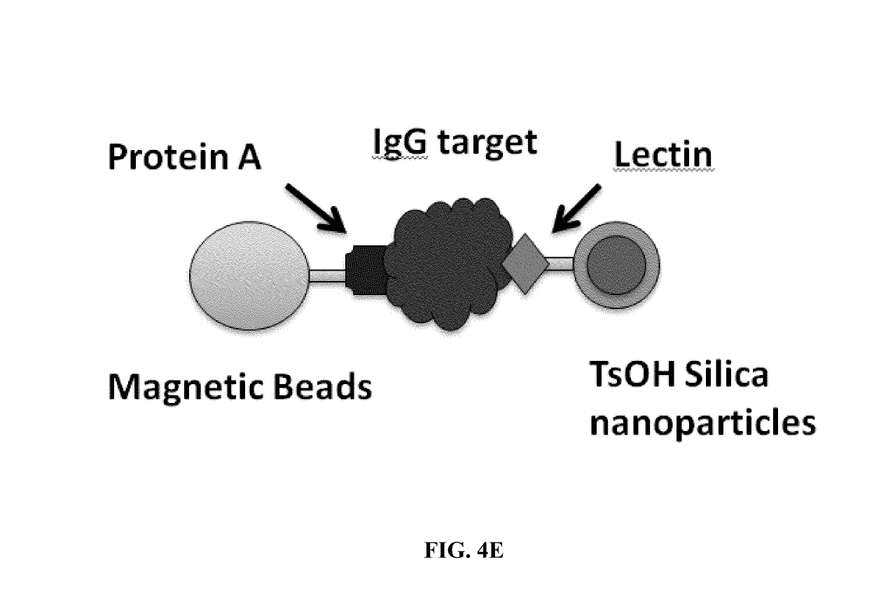

Detection of avidin protein molecules using visualization assay. The general scheme for using the assay when the desired target analyte is coupled to biotin or avidin using a solid support, is shown in FIG. 4A.

Biotin labeled silica particles were used as initiator to detect trace amount of avidin in a solution. A biotinylated glass substrate (1 inch×1 inch) was obtained by reacting the substrate with (3-aminopropyl)triethoxysilane, followed by reacting with NHS-biotin. The substrate was then placed into different concentrations of avidin solution (up to 10 pM) to capture the avidin molecules to the substrates through biotin-avidin interactions. After washing with PBS, the substrates were blocked with 1% of BSA. The substrates were then incubated with biotin labeled silica nanoparticles loaded with TsOH (0.5 mg paste/mL), followed by incubation with 10 ul of BMTA solution (10 mg/ml). After a 1.5-minute reaction, the substrates were placed in a freshly prepared solution of 15 uM methyl red. The solution without any avidin present remained yellow, the solution with 30 pM of avidin was pale pink, and the solution with 60 pM of avidin was bright red.

Detection of cancer cells through folate and folate receptor interactions. To detect cancer cells, both HeLa cancer cells and End1 normal cells were washed twice with RPMI 1640 (pH 7.2±0.2) culture media. A suspension of folate labeled silica-TsOH particles (0.5 mg paste/mL) in RPMI was added to the cell culture and incubated at 37° C. with 5% CO₂ for 2 h. Cells were then trypsinized, washed with 0.9% NaCl three times and finally suspended in 0.9% NaCl. Cells (ranging from 4000 to a single cell) were collected on a substrate disc, followed by adding 10 ul of BMTA solution (10 mg/ml) and incubated for 1.5 min. The discs were then put into the vials of methyl red indicator solution (15 uM) and the changes of the color were observed. The solutions with End1 cells present were pale peach in color (indicating no reaction occurred), and the solutions with HeLa cells present were pink.

It has been well established that HeLa cells overexpress the folate receptor because of their vast requirement for folate. As a result, the high affinity of folate for folate receptors provides a unique opportunity for the folate to function as a targeting ligand with which to associate the initiator-loaded silica nanoparticles to HeLa cells. Because of the low expression levels of folate receptors in normal End1 cells, not enough initiator-loaded silica nanoparticles were present in the End1 sample to trigger the self-catalytic reaction.

While this assay was developed by using folate conjugated silica nanoparticles loaded with an initiator molecule to detect cancer cells, the detection platform can be expanded to detect other disease biomarkers or pathogenic targets, as long as the corresponding affinity reagents are available. The expanded assays offer simple and inexpensive diagnostic methods that can rapidly detect and identify biological target analytes with minimum amounts of reagents and no instruments.

For example, when the target analyte is a nucleic acid, a complementary nucleic acid can be used as an affinity reagent. One example of such an assay to visualize the presence of a target nucleic acid analyte is depicted in FIG. 4B. An additional example is shown in FIG. 4D, using an assay to visualize the presence of an antibody-antigen interaction, which may be useful, for example, for the detection and/or identification of viruses. A further example is shown in FIG. 4E, using an assay to visualize the presence of a protein-IgG-lectin interaction, which may be useful, for example, for the detection and/or identification of Ig glycosylation levels in a patient.

Referring to FIG. 4B, a bait oligonucleotide may be used to capture the target analyte to the substrate, and a reporter oligonucleotide may be used as an affinity reagent to associate the initiator-loaded nanoparticles with the target analyte nucleic acid. The substrate can subsequently initiate the self-catalytic reaction, which will yield enough acid (pH<2) to be visualized by a pH indictor solution. Given the average number of white blood cells in 1 cc of blood to be approximately about 6 million, the concentration of genomic DNA target analyte in the sample will be in the pico to femtomolar range. The methods disclosed herein are sensitive enough for SCD detection, as described in the following prophetic example.

Atomic Force Microscopy (AFM) can be used to identify and optimize suitable capture and reporter oligo probes for SCD detection. This is especially important for the development of a reporter probe to successfully differentiate single point mutation and to reduce the chances of obtaining false positive and negative results. Although PCR may be used for the task, applying AFM and using the rapture force quantification avoids the bias intrinsic to any PCR reaction and can assess the probe performance directly and accurately.

The assay uses the change in color pattern as an indication of SCD status. The availability of the intrinsic control allows an untrained person to perform the assay with confidence. Such methods will provide a new technology for SCD diagnosis suitable for incorporation into an economical, portable “kit” for basic clinical settings and in the low-resource environments, while being robust (shelf life of BMTA has been demonstrated to be at least up to two years) and amenable to mass production.

As shown in FIG. 4C, the assay determines the zygosity of DNA by detecting a single base pair mutation (the single A-T transversion in the sequence encoding codon 6 of the human β-globin gene) that causes SCD). The detection consists of two tests, each containing a common capture oligo that captures the genomic DNA fragment to the substrate, an allele-specific reporter oligo conjugated to the initiator. The test contains a reporter oligo specific for the normal (or wild-type) DNA sequence and refractory to hybridization from mutant DNA at a given locus. Similarly, the second test contains a mutant-specific reporter oligo unable to hybridize wild-type DNA. The diagnostic conformation is achieved by analysis of the results of the assay color pattern. A normal person will be only positive (self-catalytic reactions initiated, red color) in the first test only (a color pattern of red, yellow); a heterozygote will be positive in both tests (a color pattern of red, red); and a homozygous mutant subject will be only positive in the second reaction (a color pattern of yellow, red). The color pattern of yellow, yellow can function as a control to indicate assay error.

The initiator used in this assay is core-shell silica particles, in which the core is loaded with tosylic acid (TsOH), and the shell surface is conjugated with the affinity reagent and coated with polyethylene glycol. The assay was also tested by detecting an oligo target using a pair of complementary capture and detection DNA probes. Accordingly, four oligonucleotide sequences were synthesized:

detection probe 1) NH₂-(CH₂)₆-
(SEQ ID NO: 1)
5′-TTC-TCC-ACA-GGA-3′
capture probe
(SEQ ID NO: 2)
5′-CAG-GTG-CAC-C-3′-NH₂
target-1 (disease mutation)-
(SEQ ID NO: 3)
5′-GGT-GCA-CCT-GAC-TCC-TGT-GGA-GAA-G-3′-
target-2 (normal control)-
(SEQ ID NO: 4)
5′-GGT-GCA-CCT-GAC-TCC-TGA-GGA-GAA-G-3′-

The two target sequences were based on the segments of the human hemoglobin beta chain (HBB). Target-1 is the mutant form of HBB, which contributes to the sickle cell disease; while target-2 is a normal control. Both detection and capture probes were modified with amine group so that the oligos can be anchored to the substrate and initiator, respectively. Due to the hybridization reaction, the target oligo can efficiently associate the detection probe conjugated with a silica particle initiator to the substrate via the capture probe, thus to initiate the self-catalytic reaction, while no silica particle initiator will be presented for target probe 2.

In brief, a carboxylic group modified glass substrate was used to anchor the capture probe through the use of carbonyldiimidazole to activate carboxylic acids for direct conjugation to primary amines in the oligo via amide bonds. The same chemistry was also used to link the detection probe to the silica particle initiator. The modified glass substrate, 5 nM of target oligo and modified silica particle initiator were mixed and incubated for 10 minutes. After washing the substrate with saline solution, the substrate was air dried and loaded with 10 ul of BMTA solution (10 mg/ml). After different reaction times, the substrates were placed in a freshly prepared methyl red solution. These results indicate that the methods can reliably detect the presence of disease target oligo-1 and differentiate target 1 and target 2.

Experimental design: A pair of synthetic oligonucleotides that are related to the β-globin gene in mutated and normal formats as model targets will be used to demonstrate the feasibility of the assay. Suitable oligos will then be developed and optimized as capture and reporter probes against genomic DNA fragments, and then the proof of concept of this technology against SCD using clinical DNA samples will be demonstrated.

Extend the assay to detecting synthetic oligonucleotide targets related to the point mutation of β-globin gene. The sequences of 5 single stranded DNAs are listed as the following:

Capture probe-
(SEQ ID NO: 5)
5′-GTG-TCT-GTT-TGA-GGT-TGC-TA-3′-(CH₂)₇-NH₂
Reporter probe-1-NH₂-(CH₂)₁₂-
(SEQ ID NO: 6)
5′-C-TCC-T*CA-GGA-GTC-A-3′, where T* is a
modified locking thymidine-
Reporter probe-2-NH₂-(CH₂)₁₂-
(SEQ ID NO: 7)
5′-C-TCC-A*CA-GGA-GTC-A-3′, where A* is a
modified locking adenosine-
Target 1 (Normal target)-
(SEQ ID NO: 8)
5′-TA-GCA-ACC-TCA-AAC-AGA-CAC-CAT-GGT-GCA-
TCT-GAC-TCC-TGA-GGA-GAA-GTC-TGC-C-3′-
Target 2 (Mutated target)-
(SEQ ID NO: 9)
5′-TA-GCA-ACC-TCA-AAC-AGA-CAC-CAT-GGT-GCA-
TCT-GAC-TCC-TGT-GGA-GAA-GTC-TGC-C-3′-

These sequence designs are based on the sequence of the β-globin gene. Target 1 is a 54 BP oligo, whose sequence is the same as the segment of the β-globin gene around a mutation site. The sequence of Target 2 corresponds to the segment of mutated β-globin gene responsible for SCD. A complimentary 20 bp capture probe is designed to specifically capture the target oligo. The capture probe is an amine-modified at the 3′ end with a C7 spacer. The reporter probes are each amine-modified at the 5′-end with a C12 spacer, and their sequences are complementary to a portion of Target 1 and Target 2, respectively. Target 1 differs from Target 2 by only a single base at the tenth-base position from the 3′-end. The design also includes a modified locking nucleotides in the reporter probe DNA sequences to increase the melting temperature and enhance the specificity. Specifically, reporter probe 1 includes a modified locking thymidine residue as follows:

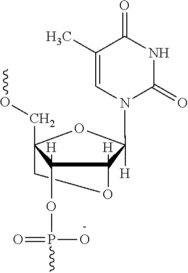

Reporter probe 2 similarly includes a modified locking adenosine residue as follows:

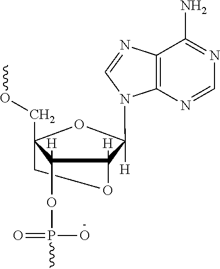

The inclusion of these modified nucleotides improves the capability of differentiating the single-base (A-T) mismatch between perfectly matched and single-base mismatched targets.

The amine modified probes will be attached to the glass substrate and silica particle surfaces using cyanogen bromide. Freshly cleaned glass will be immersed in 9.5 ml of sodium carbonate buffer of 2 M concentration. To the solution, 1 mg of cyanogen bromide dissolved in 0.5 ml of acetonitrile will be added and stirred for 2 min. An activated glass surface will be washed several times with phosphate buffer (pH 8.0) followed by DI water and placed in 5 ml of phosphate buffer (pH 8.0) to which about 420 μL of 50 μM DNA capture probe will be added. The conjugation reaction will be carried out at 4° C. for 24 hours. After 24 hours reaction, the mixture will be quenched with a large amount of phosphate buffer at pH 7.0. The capture DNA-conjugated substrate will be washed several times with the phosphate buffer at pH 7.0 and then re-suspended in about 600 μL phosphate buffer at pH 7.0 for later use. The same chemistry will be used to conjugate amine modified reporter probes to the initiator loaded silica particles coated with polyethylene glycol.

To demonstrate feasibility, we will carry out the methods by using three different experimental solutions of Target 1 alone, Target 2 alone and a mixture of Target of 1 and 2 to mimic different zygosity samples. Two tests will be conducted in parallel for each experimental solution. Each test contains the incubation of capture probe modified glass substrate, target oligo, and reporter 1 or 2 probe conjugated silica particles loaded with TSOH as initiator. The first test uses reporter 1 probe specific for the normal Target 1 and refractory to hybridization from mutant Target 2. Similarly, the second test uses reporter 2 probe, which is unable to hybridize with Target 1. In both tests, capture probe-conjugated substrate and 70 μL of reporter probe-conjugated silica particles will be mixed, followed by the addition of the target solution. The hybridization reaction will be carried out in phosphate buffer at pH 7 and 100 mM concentration of NaCl for 30 minutes. The substrate will be washed and loaded with BMTA solution, which will be subject to initiate the self-catalytic reaction. Experimental solution 1 (Target 1 alone—normal) will be only “positive” (self-catalytic reaction occurs, red color) in the first test (a color pattern of red, yellow); Experimental solution 2 (Target 1 and 2 mixture—heterozygote) will be positive in both tests (a color pattern of red, red); and Experimental solution 3 (Target 2 alone—homozygous mutant) will be only positive in the second test (a color pattern of yellow, red). A color pattern of yellow, yellow can function as a control to indicate assay error.

Develop and optimize capture and reporter oligo probes against genomic DNA fragment for the colorimetric visualization assay. Genomic DNA target preparation: Whole genomic DNA from normal human End1 epithelium cells (confirmed with no β-globin gene mutation by PCR) will be used initially to develop suitable capture and reporter probes. A combination of two different restriction enzymes to digest the genomic DNA for obtaining the target DNA segments will be used.

A combination of Bfal and MaeIII enzymes will be used to digest the genomic DNA to obtain the same 54 bp DNA segment as used as the detection target, which will include the mutation site of β-globin gene. Both capture and reporter probes will be developed against pure Target 1, genomic DNA segments from End1 cell, pure Target 2, and a mixture of Target 2 with whole genomic DNA from End1 cells.

Briefly, 0.5 ml of End1 epithelium cell suspensions will be treated with a cold lysis buffer, containing 0.2 g Tris, 21 g sucrose, 0.2 g MgCl₂, and 2 mL Triton 100× in 200 mL of distilled water to disrupter cells and nuclear membranes to release cellular DNA. The precipitate will be washed three times and genomic DNA will be extracted by adding 100 mL of 50 mM NaOH. Subsequently, the tube will be heated in a boiling water bath to solubilize the DNA. Alternatively, DNA can be isolated from the blood by use of the InstaGene™ Whole Blood Kit (Bio-Rad Labs. Hercules, Calif.). The purified genomic DNA will be digested with BfaI and MaeIII restriction enzyme mixture at elevated temperature (35-40° C.). DNA fragments will be denatured by 5-min incubation at 95° C., followed by fast cooling on ice to maintain as single strand fragments.

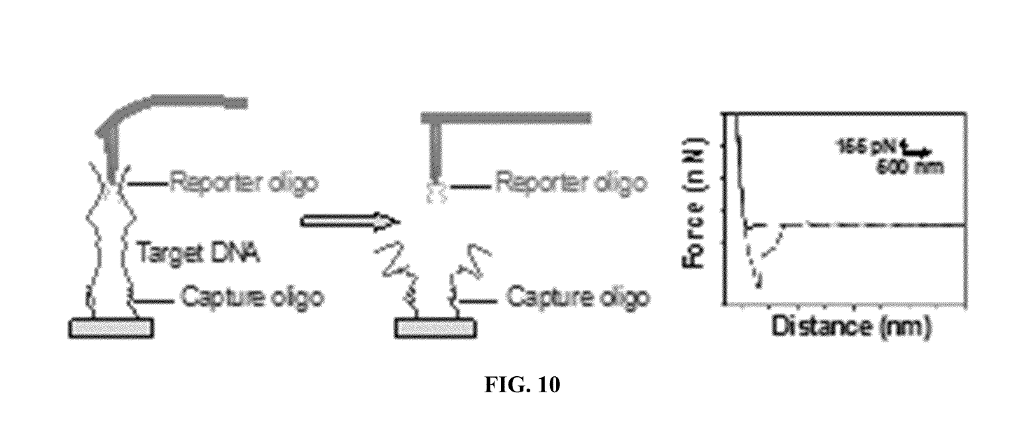

Because of the presence of a large amount of different genomic DNA fragments after enzyme digestion, a key task is to identify and optimize the right oligos to function as capture and reporter probes for the VA assay, thus reducing unwanted hybridization while ensuring the reporter probe can differentiate the desired SNP. This will be achieved by quantifying the binding strength of different reporter oligos against target DNA sequence using an AFM. The method anchors the capture probe on a surface and attaches the reporter probe on an AFM tip (See FIG. 10).

Probe performance against the target DNA sequence is assessed by the hybridization events among the complimentary strands, which are transduced by an adhesive force during the AFM tip approaching and retracting from the surface. The adhesion force from an oligo-dT modified tip and an oligo-CD44 (5′-HS-GAATGTGTCTTGGTCTCTGG (SEQ ID NO: 10), containing the sequence of cDNA of CD44) modified surface was obtained, which were bridged by mRNA then retrieved from cells. An initial adhesion force of 0.35 nN was observed, which diminished in the following cycles of measurement. The interchain force between a pair of complementary DNA strands (20 bases in length) was reported at ˜1.5 nN. Since the interaction of dA-dT is much weaker than that of strands involving C-G interaction, the 0.35 nN force is likely associated with the permanent dissociation of the dA-dT hybrid, which can be well detected above the non-specific interaction forces in the range of several tens of pN. This method will be used to evaluate both capture and reporter probes and to optimize the number of LNA and their position for the reporter probe.

Experimentally, a minimum of 20 bases will be used for the capture probe and a minimum of 14 bases for the reporter probe to ensure the strong and specific interaction between the probe and the target DNA strand. The reporter oligo will be modified with a C12-thiol group at its 5′ terminal for its conjugation to the gold-coated AFM probe; the capture oligo will be modified with a C7-amine at its terminal for its conjugation to the glass substrate using cyanogen bromide chemistry. These pre-modified oligos can be commercially synthesized. After incubating modified AFM tip and substrate with genomic DNA fragments, force curves during AFM tip approaching and retraction from the surface will be recorded to provide quantitative evaluation of binding affinity of the candidates. Force measurements will be carried out at five different locations and 20 curves per location, warranting the statistical significance of the data. Note that the same capture oligo modified substrate will be used to screen all reporter oligos. Consistent AFM probe modification protocols will be utilized. Thus the difference observed in force measurement reflects the binding strength of the reporter oligo regardless of the number of oligos per tip. Corresponding adhesion force for different capture and reporter probes will be recorded. By changing the sequence of the reporter probes as well as the number and location of LNA modification, probes with maximum adhesion force that are also capable of differentiating the desired SNP will be used. Genomic DNA from normal and SCD patients will be used to validate the probes.

In case the hybridization between the DNA fragment and probes is not effective due to immobilization of the probes, fluorescence cross-correlation spectroscopy (FCCS) can be used as an alternative method to select and optimize the probes. By labeling the capture and reporter probes with Alexa 488 dye and Alexa 632 respectively, the hybridization of both probes with the target can be followed by FCCS. In addition, the appearance of PCR products also can be used to evaluate the probe performance by using the probe oligos as the primers.

Demonstrate the proof-of-concept for a colorimetric visualization assay for differentiating normal (HbA), sickle cell trait (HbAS) and sickle cell anemia (HbSS) at the genomic level. Whole blood samples will be obtained. These samples will be from unrelated sickle cell disease patients whose hemoglobin genotype have been confirmed in the lab using hemoglobin HPLC and family history. Patients and normal controls are randomly selected from both males and females, to include patients with and without blood transfusion. Sickle trait samples will also be obtained from parents of the patients with SCD, as well as babies referred for newborn screening detection of sickle trait. After thawing, 500 μL of EDTA-treated sample will be lysed and digested by the combination of Bfal and MaeIII restriction enzymes using the same method outlined above. The actual sample treatment time will depend on the enzymes, enzyme concentrations, temperature and buffer composition and can be done in less than 15 minutes.

To demonstrate the proof-of-concept, two tests will be conducted in parallel for each clinical sample in different zygosity using the protocols outlined above 1. Each test contains the incubation of capture oligo modified glass substrate, whole genomic DNA fragments, and reporter oligo conjugated silica particles loaded with TSOH initiator. The diagnostic confirmation is achieved by analysis of the results of visualization assay color pattern. A normal person should be only positive (self-catalytic reaction occurs, red color) in Reporter Probe 1 test (a color pattern of red, yellow); a trait person will be positive in both tests (a color pattern of red, red); and an SCD patient should only be positive in Reporter Probe 2 test (a color pattern of yellow, red). A color pattern of yellow, yellow can function as control to indicate assay error. Both specificity and sensitivity of the assay will be benchmarked with different clinic samples. With a two-minute reaction time, the tests can be finished in 15 minutes, yielding a conservative total assay time of 30 minutes.

Due to the complexity of clinical DNA samples, especially the fact that sickle red blood cells may contain 20% more RNA nuclide materials due to the presence of immature red blood cells in SCD, the assay may experience a higher rate of false positives. The SfaNI enzyme has a recognition site four base from the mutation site, and cleaves both normal and mutated DNA into two fragments, yielding both reporter probes not working anymore. By including additional tests using SfaNI and reporter probes, the combined results will significantly reduce the false positive rate. Any other color combinations/patterns will indicate assay error.

Referring to FIG. 4E, a bait protein may be used to capture the target analyte to a magnetic substrate, and a reporter protein may be used as an affinity reagent to associate the initiator-loaded particles with a target analyte immunoglobulin. The substrate can subsequently initiate the self-catalytic reaction, which will yield enough acid (pH<2) to be visualized by a pH indictor solution. The methods disclosed herein are sensitive enough for the detection of immonglobulin glycosylation levels, as described in the following example.

In the human immune system, all immunoglobulins (Ig) are glycosylated. It has been demonstrated that the change of Ig glycosylation level is associated with variety of diseases including cancer, rheumatoid arthritis, acquired immune deficiency syndrome (AIDS), inflammatory bowel disease, periodontal disease, Crohn's disease, tuberculosis and infection with HIV. Studies have also shown that the level of Ig glycosylation is a potential biomarker for cancer diagnosis. A simple visualization assay to disclose the level of Ig glycosylation in a patient's serum provides an economic, yet effective detection method for cancer screening and early diagnosis.

As depicted in FIG. 4E, the assay involves the use of magnetic beads conjugated with protein A as the substrate and TsOH-silica particles anchored with lectin as the initiator. Protein A specifically interacts with IgG, and lectin is a carbohydrate-binding protein that is highly specific for the sugar moieties of a glycoprotein including IgG. When mixing a sample containing IgG with magnetic beads conjugated with protein A and TsOH-silica particles anchored with lectin, the level of glycosylation in IgG determined the number of TsOH-silica particles (initiator) bound to the magnetic bead substrate. After separating any unbound TsOH-silica particles using a magnetic separator, the magnetic beads were mixed with BMTA to initiate the self-catalytic reaction and visualized in 2 mL of a methyl red (15 μM) indicator solution. The results showed that both the blank samples and control samples remained yellow, and that the samples with the magnetic beads were bright pink. The assay was able to effectively detect the presence IgG (3 nM) in solution, while control experiments of magnetic beads themselves (control) or containing album protein molecules (blank) did not show any color change.

The above assay may be adapted for the detection of viruses such as HIV, HPV, West Nile, and Ebola, because the envelope proteins of all these virus contain glycoproteins. By using magnetic beads conjugated with an antibody specific to a particular envelope virus and TsOH-silica particles anchored with lectin, the presence of virus in a patient's serum can be detected in a similar way.

### Example 2

### Exemplary Bioassays Based on a Target Analyte—Initiator Association Through Physical Absorption of the Initiator to the Target

The assay involves a self-catalytic system that yields a color change in a pH indicator solution based upon the existence of cancer cells in a mixed cell sample. The presence of enlarged nuclei in cancer cells leads to the introduction of initiator molecules, which bind tightly to the nuclei of the cancer cells, to the self-catalytic system in an amount above the threshold to initiate the self-catalytic reaction. The self-catalytic reactions yielded enough acid (pH<2) to be visualized by a pH indictor solution. At least a billion-fold of signal amplification can be achieved within 2-5 minutes. The assay was capable of detecting the presence of cancer cells in a mixed cell sample which contained 20% cancerous cells.

Unlike the Pap smear bioassay, which is based on reviewing and analyzing the characteristic morphology of nuclei in abnormal (cancerous) cells under a microscope, this assay differentiated the amount of nuclear material in cancerous cervical cells as compared to normal cervical cells. Such a “cancer litmus strip” approach required minimal reagents, no instruments, is simple and fast, and could potentially allow physicians to make decisions regarding additional cancer screening within minutes, thus improving screening programs against cervical cancer and HPV in low-resource environments. The present example demonstrates the development of a colorimetric assay for sensitively detecting and potentially quantifying cancer cells by simple visualization.

Visualization bioassay. HeLa (ATCC, Manassas, Va.) cells were maintained in DMEM+10% FBS, 5% CO₂ and humidified atmosphere. Growth medium for End1 (ATCC, Manassas, Va.) was keratinocyte-SFM 1× supplemented with L-glutamine, human recombinant EGF, bovine pituitary extract and calcium chloride. Not more than 10 passages of cells were used throughout the experiment. When the flasks reached 90% confluence, cells were harvested using TrypLE (Gibco, Grand Island, N.Y.) according to the manufacturer's protocol. Harvested cells were suspended in saline to a concentration of 100,000 cells/mL. A 50 μg/mL Lillie-Mayer solution was added to the cells and incubated at room temperature for 10 minutes. The stained cells were washed with 0.9% NaCl and suspended to a concentration of 500 cells/μL. About 2000 cells were adsorbed on a substrate disc (diameter 7.0 mm) and incubated for 5.0 minutes at room temperature. BMTA (4.0 μL, 28.06 mM) was absorbed onto the cells, followed by treating the loaded disc for 1.5 min. The substrate disc was placed into a glass vial and 2 mL of methyl red (15 uM) indicator solution was added to observe a color change.

Results

Sensor principle. As noted in FIG. 1, the visualization assay consisted of a self-catalytic system initiated by a trace amount of an acidic initiator. In some embodiments, the initiator is specifically introduced to the system due to the presence of a biomarker or disease molecule or cell itself (e.g., cancer cells with acidic nuclei or acid staining media added). After the self-catalytic reaction cycles, the formation of p-toluene sulfonic acid (TsOH) can be visualized by a pH indicating dye, which in turn, indicates the presence of the target analyte, that being the biomarker or disease molecule/cell.

One method of detecting the presence of abnormal precancerous or cancerous cervical cells is to use a Pap smear test. In the Pap smear test, cells are stained with hematoxylin and examined under a microscope to look for cellular abnormalities, usually of enlarged nuclei. In hematoxylin staining, hemalum (a hematein-alumina complex), the active ingredient in the staining solution, binds tightly to lysine and arginine residues of nuclear histones. Since histones are extremely rich in the nuclides, the hemalum specifically associates with and stains cell nuclei, which can be visualized under a microscope. Abnormal cells, including cancerous cells, typically have nuclei that are larger than that of normal or benign cells, and are known to develop even larger nuclei as they become more malignant. Due to the high abundance of nuclear histones and more DNA in their enlarged nuclei, stained abnormal and/or cancerous cells possess much more hemalum compared to the normal or benign cells. Structurally hemalum is an organic acid due to the presence of the hydroxyl chromene groups:

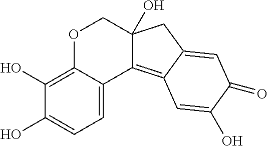 [SMILES: O=C1C=C2CC3(O)COc4c(ccc(O)c4O)C3=C2C=C1O]

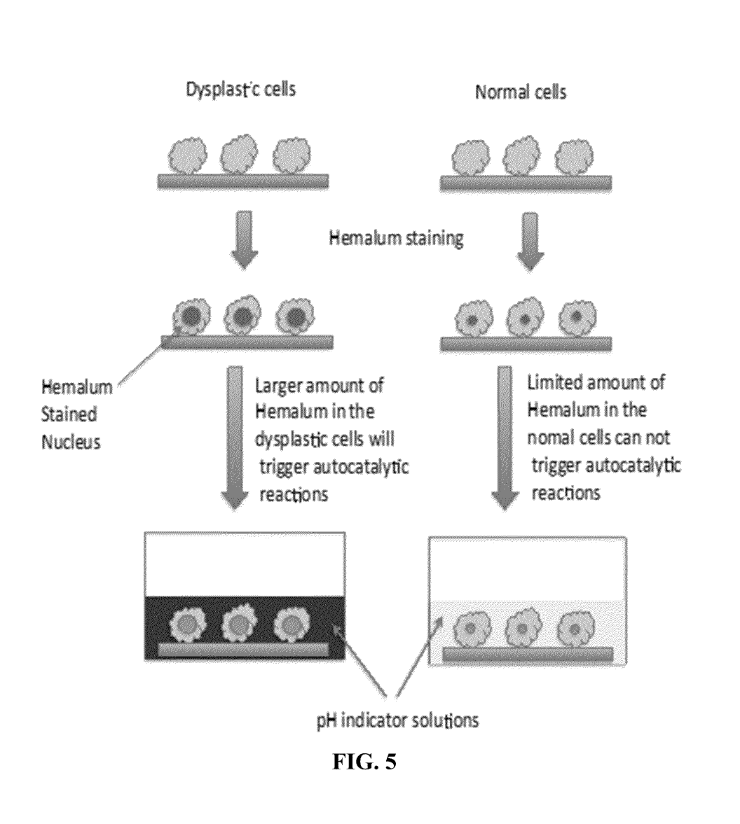

Hemalum can specifically bind to cell nuclei; thus the use of hemalum as an initiator for BMTA self-catalytic reaction was explored (FIG. 5). The relatively large amount of hemalum present in the nuclei of stained abnormal cells is able to initiate self-catalytic reactions, while the limited amount of hemalum associated with the normal cells cannot, or takes a much longer time to, initiate the self-catalytic reaction. Acid generated from the reaction can trigger the change of a pH-sensitive indicator dye, such as changing the yellow solution of methyl red to a red solution, making the presence of cancerous cells visible by a color change. This is a so-called “cancer litmus strip test” approach.

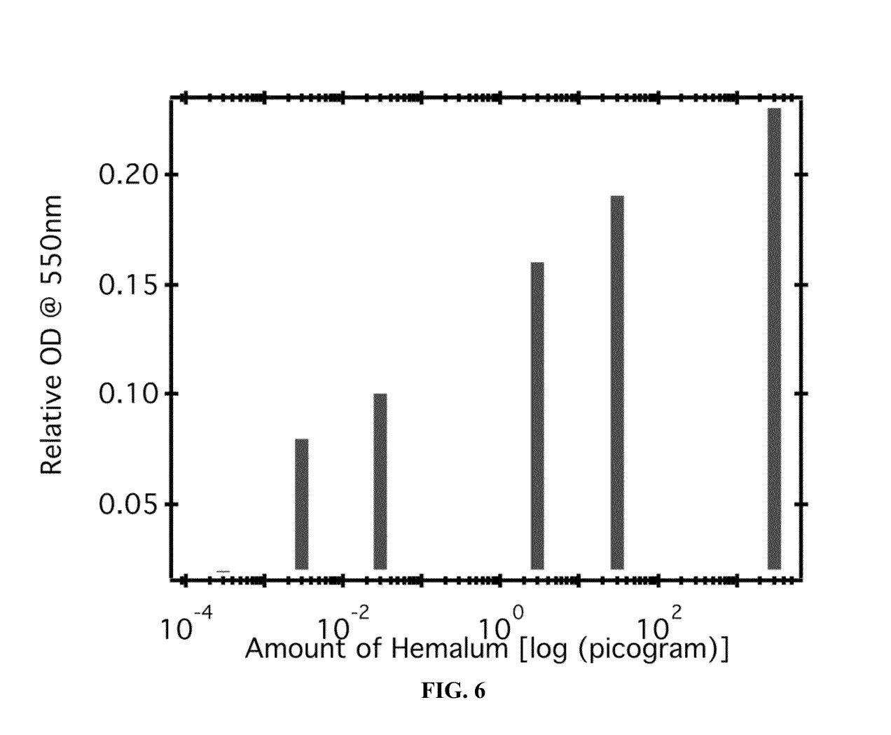

Hemalum can function as an initiator/catalyst to initiate the self-catalytic reaction. As shown in FIG. 6, as little as 3 femtogram of hemalum can initiate the self-catalytic reaction and produce enough protons in 1.5 minutes to induce the color change in a methyl red solution. The color change can be easily seen and detected with the human eye. FIG. 6 further illustrates the optical densities at 550 nm of the methyl red solution after different self-catalytic reactions as the function of hemalum amount (in log scales). The amount of hemalum initiator in the log scale is linearly proportional to the proton concentrations, which in consistent with the self-catalytic reaction mechanism. The results suggest that the assay has the potential to quantitatively analyze the amount of hemalum present in a solution, which is proportional to the number of hemalum stained cells.

Additional experiments were performed to more fully explore the correspondence between the amount of hemalum used to initiate the self-catalytic reaction and the resultant color change. The cells were treated as follows. All cell lines were cultured in an incubator at 37° C. with a mixture of air and 5% CO₂. HeLa (ATCC, Manassas, Va.) cells were maintained in in DMEM (GIBCO) supplemented with 10% (v/v) fetal bovine serum (Irvine Scientific). End1 (ATCC, Manassas, Va.) cells were maintained in keratinocyte-SFM (GIBCO) supplemented with L-glutamine, human recombinant EGF, bovine pituitary extract (GIBCO) and calcium chloride. Cells from no more than 10 passages were used throughout the experiments. When the flasks reached 90% confluence, cells were harvested using TrypLE (Gibco, Grand Island, N.Y.) according to the manufacturer's protocol. Briefly, cells were rinsed using 1×PBS to remove residual growth media and incubated with TrypLE for 2 min at 37° C. Collected cells were then centrifuged at 1100 rpm for 8 min in an Eppendorf 5810R centrifuge (Westbury, N.Y.) at 4° C. The pellets were suspended in PBS and counted on a hemacytometer (Hausser Bright-Line, Fisher Scientific). The suspended cells were washed two times with cold PBS buffer and finally re-suspended in 0.5 mL cold PBS. Cells were fixed by carefully mixing the suspension in 4.5 mL cold absolute ethanol and incubating for 2 min at 4° C. After washing the fixed cells with PBS followed by one time wash with 0.9% NaCl, the hemalum solution (50 μg/mL) was added to the cell pellets and incubated for 2 min at room temperature. The stained cells were washed with 0.9% NaCl and suspended to a concentration of 1 cell/2 μL for the assays.

To quantify the amount of hemalum in HeLa and End1 cells using HPLC, the following procedure was used. Trypsinized HeLa and End1 cells were washed and suspended in 0.5 mL cold (4° C.) PBS. The cell suspension was mixed with cold (−20° C.) ethanol (4.5 mL) and incubated at 4° C. for 2 min. After washing the fixed cells with PBS followed by one time wash with 0.9% NaCl, 5.0 mL of hemalum solution (50 μg/mL) was added to the cell pellets and incubated for 2 min at room temperature. The stained cells were washed with 0.9% NaCl and suspended to a final concentration of 6×10⁵ cells/vial for optical imaging.

To extract hemalum from the cells, 1 mL of 1% HCl in 70% EtOH was added to the cell plates and the cell suspension was probe-sonicated for 6 min to yield a homogeneous purple-red mixture. The resulting solution was filtered through a syringe filter with a cellulose acetate membrane (0.2 micrometer pore size) to remove the cell debris. 20 μL of the filtrate (equivalent to the amount of hemalum in ˜12000 cells) were manually injected into an HPLC (Agilent 1200) equipped with a Zorbax Rx C-18 column. The elution solvent was isocratic 5% acetonitrile in water with 0.1% TFA. A characteristic peak of hemalum at 8.75 min was detected using a UV-V is diode array detector at a 350 nm wavelength. A standard working curve was prepared using the known concentrations of hemalum dye to quantify the amount of hemalum present in HeLa and End1 cells.

For the assays, 2 μL of stained cell solution was loaded onto a substrate. The presence of a single cell on the substrate was confirmed using an Olympus stereomicroscope. Subsequently, 4 μL of BMTA solution in acetonitrile (10 mg/mL) was added to the substrate. The substrate was then incubated at 85° C. for 1.5 minutes and consequently placed into a vial of freshly prepared yellow indicator solution of methyl red. The change of color of the indictor solution was observed by eye and the optical density of the indicator solution was quantified using a Uv-Vis spectrometer.

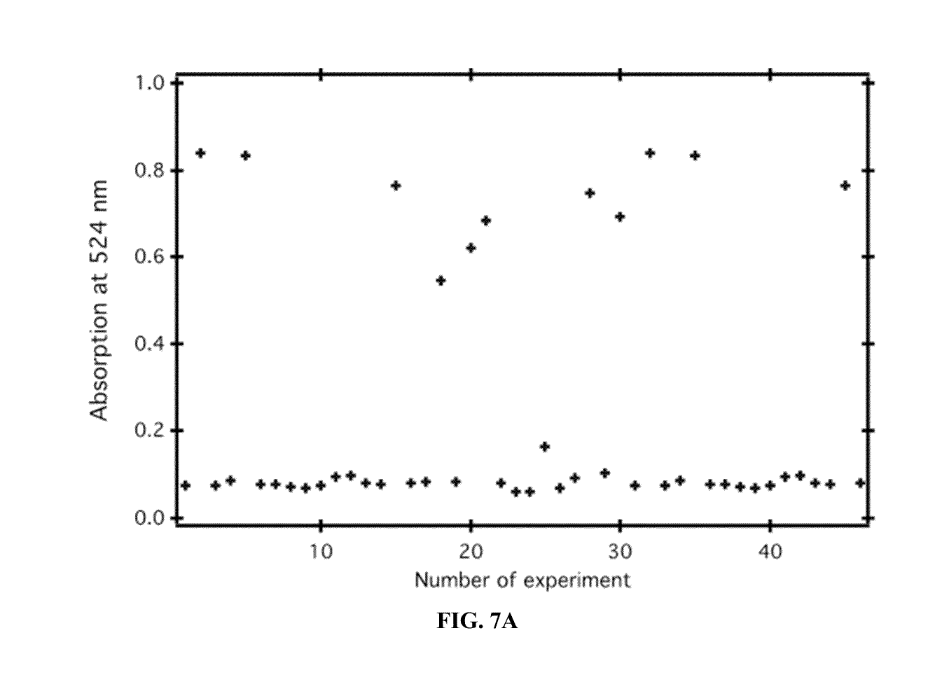

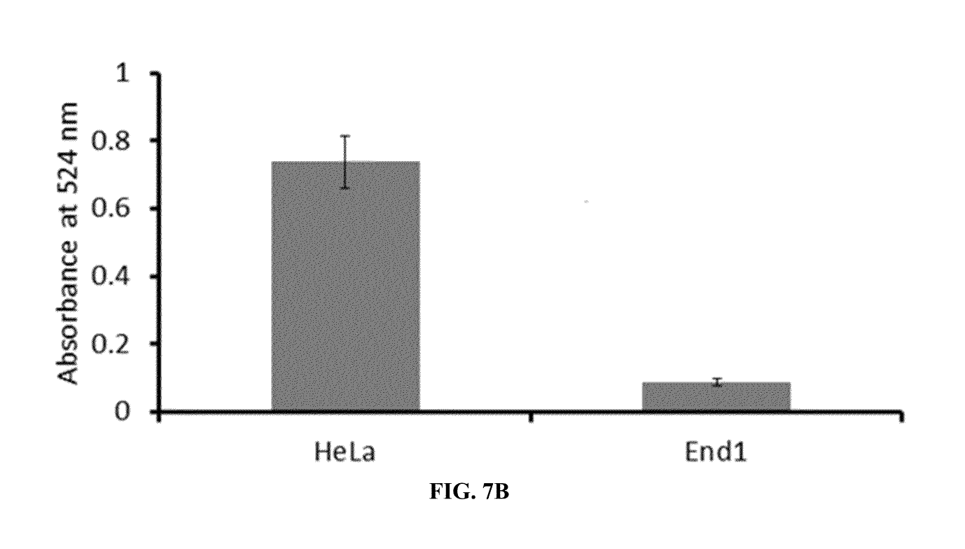

Hemalum stained HeLa and End1 cells were mixed with 80% of End1 and 20% of HeLa cells, judged by the hemocytometer. The cell mixture was then diluted to the level of 1 cell/2 μL. 2 μL of the cell suspension was loaded onto the surface of the substrate and the presence of single stained cell on the substrate was again confirmed by the microscope. A total of 45 assays were performed using the cell mixture, with the results shown in FIG. 7A.

Stained HeLa and End1 cell suspensions were diluted to the level of 1 cell/2 μL. 2 μL of diluted cell suspension were loaded onto the surface of the substrate and the presence of a single stained cell on the substrate was confirmed by examining the substrate under a microscope with 40× objective lens. Subsequently, 4 μL of BMTA solution in acetonitrile (10 mg/mL) were added to the substrate. The substrate was then incubated at 85° C. for 1.5 minutes and subsequently placed into a vial of freshly prepared yellow indicator solution (methyl red turned yellow at pH 8.0). All experiments with stained HeLa cells exhibited a color change from yellow to pink-red while the experiments using stained End1 cells remained yellow, indicating that no self-catalytic reaction had occurred within 1.5 minutes for the End1 cells. The experiments were repeated hundreds of times with different batches of cells, and similar results were obtained. The absorbance of the resulting indicator solution was measured by UV-Vis, and the corresponding optical densities at 524 nm were plotted for both HeLa and End1 cells (FIG. 7B).

To test the use of hemalum to catalyze the reaction of BMTA, different amounts of hemalum (in picogram range) were mixed with BMTA in acetonitrile (0.11 μmole) and loaded on different substrates, each of which was heated at 85° C. for 1.0 minute. The substrate was then placed in a freshly prepared methyl red solution and the color change of the solution was visualized. The optical density of the resulting Methyl Red solution was further quantified using a UV-Vis spectrometer.

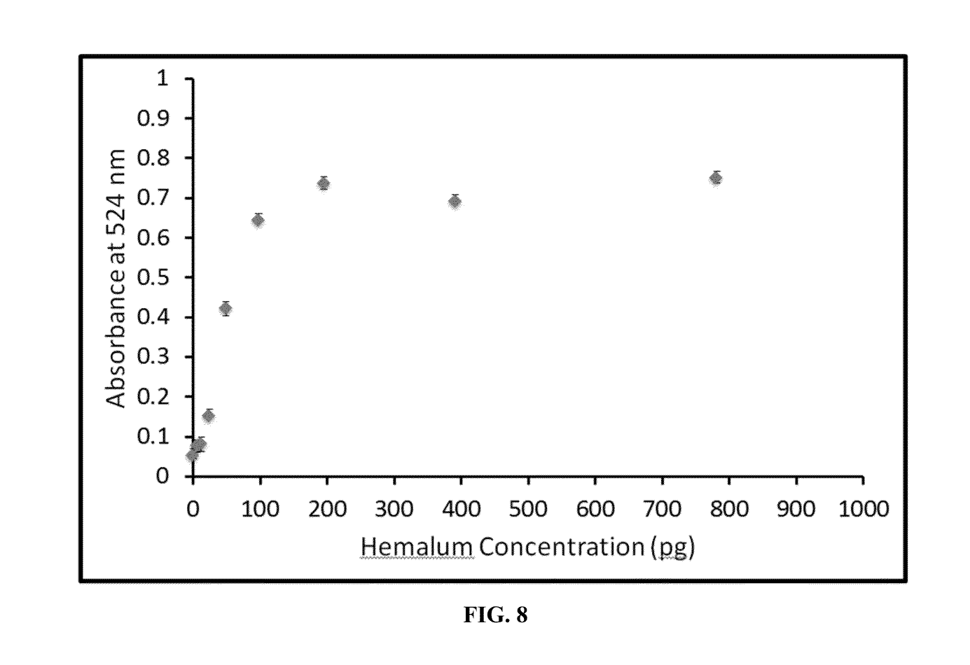

FIG. 8 illustrates the change in optical densities of the methyl red solutions resulting from the self-catalytic reaction in response to different amounts of hemalum as the initiator. Approximately 200 picograms of hemalum can initiate the self-catalytic reactions to produce enough protons in 1 minute to saturate the color change in the methyl red solution from yellow to pink-red, which can be easily seen by the human eye. The sigmoid shape of the plot indicated that acid production increased progressively as the amount of hemalum (initiator) increased, again supporting the self-catalytic reaction mechanism.

Visualization assay to detect cancer cells. Cancerous Hela and Skov-3 cells were used as positive samples and normal endocervical epithelial End1 cells were used as the negative control sample. Both cancerous and normal cells were trypsinized and detached from the culture dish. The suspended cells were washed two times with 0.9% NaCl and finally suspended in 0.9% NaCl. Hematoxylin solution (0.8 mM final concentration) was added to the cell suspension and incubated for 10 min at room temperature. The stained cells were collected and wash twice with saline. Both the cancerous and normal cells were imaged under a bright-field microscope to confirm that the cancerous cells had enlarged nuclei and thus stained with more hemalum compared to the normal cells (data not presented). Approximately 2500 cells were collected on the substrate disc, fixed with 95% alcohol and washed with water. Subsequently, the cells were then incubated with BMTA in acetonitrile for 5 minutes. The substrate disc was then treated for 1.5 min and consequently placed into a vial of indicator solution (methyl red) to yield a visible color change of the solution from yellow to pink/red. Specifically, the samples containing HeLa and SKOV-3 cells showed a color change from yellow to red, while the sample containing stained End1 cells was found to remain yellow in color, indicating that no self-catalytic reaction occurred within 1.5 minutes. The experiments were repeated at least 20 times with different batches of cells, and similar results were obtained. Similar results were also obtained when using 100-1000 cell sample sizes.

To explore the sensitivity of the methods, the assay was performed using a mixture of cancerous cells and normal cells. The assay was performed using a mixture of cancerous and normal cells with a fixed total number of 1000 cells. The assay reliably detected the presence of cancerous cells in a mixture of 20% of cancerous cells and 80% of normal cells by showing a red color, wherein 100% End1 cells remained yellow.

In certain embodiments, the presence of the enlarged nuclei of cancerous cells introduces initiator molecules in an amount above the threshold, and can initiate the reaction. This subsequently triggers the self-catalytic reactions to generate enough acid (pH<2) to be visualized by the pH indicator solution. At least a million folds of amplification can be achieved within 2-5 minutes, and is capable of detecting at least about 20% cancerous cells in a cell mixture.

Theoretical sensitivity. In general, the human eye can easily distinguish a 5% change in the color of solution. For a colorant with a relatively high extinction coefficient (for example, 8=50,000), such as the methyl red pH-dependent indicator, the number of new colorant molecules needed to induce a distinguishable color change in a fixed volume of solution (using a 1 cm path length and 1 ml volume) will be 10¹⁴ molecules based on the Beer-Lambert law {Log(100/95)=50,000×1×[colorant]}. Considering the reaction efficiency between the pH indicator and proton (1:1), one may conservatively estimate that about 10¹⁷ protons in 1 mL of solution are needed to induce a visible color change. When staining a cell with initiator, approximately 1-1000 initiator molecules (equivalent to approximately 1−1×10³ protons) can be estimated to be present in a single target analyte (i.e. in the cell). Thus an amplification step to produce an additional 10¹⁴-10¹⁷ protons is needed. This can be feasibly achieved with the acid self-catalytic reactions using 10¹⁵-10¹⁸ BMTA molecules (approximately about 3 mg BMTA). Thus, the assay has the potential to detect a single target molecule. As the amount of hemalum present in the assay, which is proportional to the number of cells, determines the self-catalytic reaction rate, the color intensity or the time to observe the color change may correlate with the number of cancerous cells present in the sample. Using a standard working curve (plotted against a known number of cancerous cells), the assay could quantify the number of cancerous cells present in a sample.

An additional set of experiments were performed to quantify the amount of hemalum associated with stained cells. HeLa cells were used as a positive cancer cell model, while normal endocervical epithelial End1 cells were used as a negative control sample. After the cells were fixed with absolute ethanol for 2 min, hemalum solution (50 μg/mL) was added to the cell suspension and incubated for 2 min at room temperature. The stained cells were collected, washed twice with water, and placed on a slide for optical imaging. The bright field microscope images revealed that the nuclei of HeLa cells were larger than those of End1 cells, suggesting that HeLa cells host more haemalum compared to End1 cells.

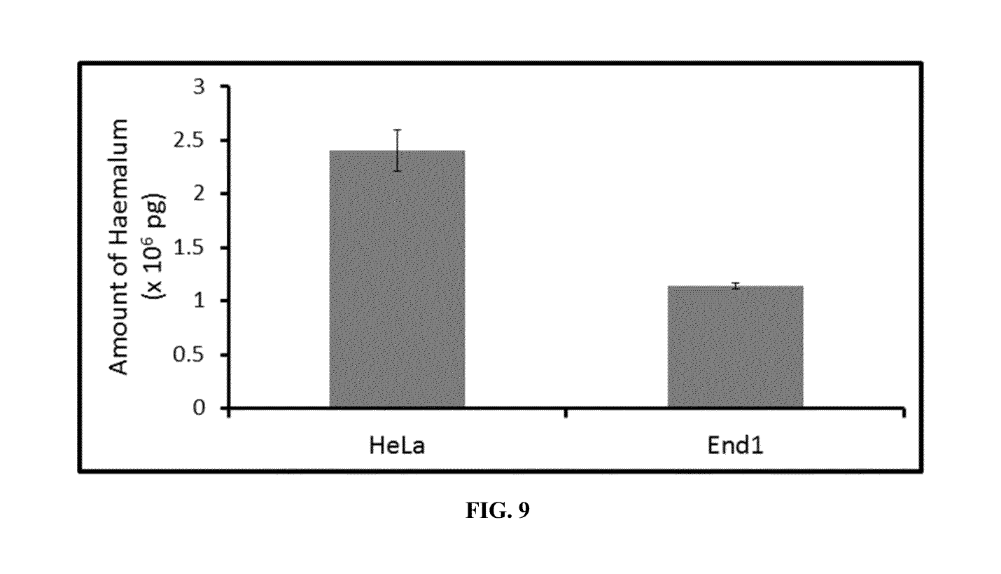

To further quantify the amount of hemalum associated with the stained cells, hemalum was extracted from the cells by mixing the cells with acidic ethanol (mixture of 1% HCl with 75% of ethanol) and probe-sonicating them in ice for 6 minutes. Cell debris was removed by filtration, and the resulting clear solutions were subjected to quantitative analysis. The amount of hemalum was determined by high-performance liquid chromatography (HPLC) equipped with a C18 reversed-phase column (Zorbax Rx C-18) using an isocratic solvent system of 5% acetonitrile in water acidified by 0.1% TFA. Hemalum exhibits an elution peak with a retention time of 8.75 minutes. By analyzing the peak areas of hemalum from 12,000 HeLa and End1 cells (determined by a hemocytometer), respectively, and comparing them to the standard working curve, we estimated that each HeLa cell hosted approximately 201 picograms of haemalum while approximately 95 picograms of haemalum resided in each End1 cell, as shown in FIG. 9. This confirmed that a stained cancer cell possesses twice as much haemalum molecules as a stained normal cell.

### Example 3

### Exemplary Bioassay Based on a Target Analyte, which can Function as an Initiator Itself, Initiated Self-Catalytic Reaction

The acidic nature of cancerous cell facilitates the detection of cancer cells. It has been established that cancerous cells are overall more acidic than normal cells due to the “Warburg” effect, in that cancer cells preferentially convert glucose and other substrates to lactic acid (pKa=3.7) to produce more ATP to accommodate their uncontrolled growth. Such a feature of enhanced proton extrusion and lactate retention for cancerous cells can be beneficial for a visualization assay for cancer cell detection. The presence of 2000-5000 HeLa cancer cells without hematoxylin staining could theoretically trigger the self-catalytic reaction of BMTA while the same amount of normal cells cannot.

The present disclosure provides for an assay that is suitable for incorporation into economical, portable devices for very basic clinical settings, while being robust and amenable to mass production. It offers unconventional, creative, and bold methods useful in screening programs against such diseases as cancer, HPV, AIDS, and SCD.

What is claimed is:

1. A method for determining the presence or concentration of an analyte in a sample, comprising:
associating an initiator with the analyte to form an analyte associated initiator,
contacting the analyte associated initiator with a reagent adapted to undergo a self-catalytic reaction when contacted with the initiator, wherein the self-catalytic reaction produces a product, and
obtaining an observation or measurement corresponding to a change in the amount of the product.

2. The method of claim 1, wherein the analyte is selected from at least one of a cell, virus, protein, hormone, antibody, antigen, receptor, ligand, polynucleotide, peptide, or other biological molecule.

3. The method of claim 1, wherein the reagent is an acid amplifier.

4. The method of claim 3, wherein the acid amplifier is selected from at least one of tert-butyl 2-methyl-2-(tosyloxymethyl) acetoacetate, pinanediol monotosylate, a benzyl sulfonate, an acetoacetate derivative, an alpha-ketal sulfonate, a 1,2-diol monosulfonate, a trioxane derivative, a cyclohexane-1,4-disulfonate, or a polymer-bound pinanediol monosulfonate.

5. The method of claim 1, wherein the initiator is an organic acid.

6. The method of claim 5, wherein the organic acid is selected from at least one of dodecyl p-toluenesulfonate, ethylene di(p-toluenesulfonate), 2-butynyl p-toluenesulfonate, phenyl p-toluenesulfonate, tetrasodium pyrophosphate, or p-toluenesulfonic acid.

7. The method of claim 3, wherein the analyte is a cancerous or abnormal mammalian cell that has an enlarged cell nucleus relative to a normal cell, and the initiator is an acidic cell staining agent.

8. The method of claim 7, wherein the acidic cell staining agent comprises hemalum.

9. The method of claim 1, wherein the initiator is associated with a binding moiety adapted to specifically bind to the analyte.

10. The method of claim 9, wherein the binding moiety comprises an antibody, antigen, polynucleotide, protein, or small molecule.

11. The method of claim 9, wherein the binding moiety is coupled to a carrier infused with the initiator.

12. The method of claim 11, wherein the carrier comprises a porous silica particle.

13. The method of claim 1, further comprising binding the analyte to a solid support.

14. The method of claim 13, wherein the solid support comprises a binding moiety attached thereto, the binding moiety adapted to specifically bind the analyte.

15. The method of claim 1, further comprising comparing the observation or measurement associated with the sample to an analogous observation or measurement from at least one control sample, and determining the presence or concentration of the analyte in the sample based on the comparison.

16. The method of claim 1, wherein the product comprises a proton, and wherein obtaining an observation or measurement corresponding to a change in the amount of the product comprises visualizing a color change of a pH-dependent indicator.

17. A kit for detecting an analyte, comprising:
an initiator,
an associating agent adapted to associate the initiator with the analyte to form an analyte associated initiator,
a reagent adapted to undergo a self-catalytic reaction when contacted with the initiator, and
instructions for their use.

18. The kit of claim 17, wherein the reagent is an acid amplifier.

19. The kit of claim 18, wherein the acid amplifier is selected from at least one of tert-butyl 2-methyl-2-(tosyloxymethyl) acetoacetate, pinanediol monotosylate, a benzyl sulfonate, an acetoacetate derivative, an alpha-ketal sulfonate, a 1,2-diol monosulfonate, a trioxane derivative, a cyclohexane-1,4-disulfonate, or a polymer-bound pinanediol monosulfonate.

20. The kit of claim 17, wherein the associating agent comprises a binding moiety adapted to specifically bind to the analyte.

21. The kit of claim 20, wherein the binding moiety is coupled to a carrier adapted to be infused with the initiator.

22. A kit for detecting cancerous or abnormal mammalian cells that have an enlarged cell nucleus when compared to normal cells, comprising:
hemalum,
tert-butyl 2-methyl-2-(tosyloxymethyl) acetoacetate, and
instructions for their use.


SEQUENCE LISTING (10 sequences)

SEQ ID NO: 1 - DNA, 12 bp, Artificial Sequence
  Synthetic Oligonucleotide
ttctccacagga

SEQ ID NO: 2 - DNA, 10 bp, Artificial Sequence
  Synthetic Oligonucleotide
caggtgcacc

SEQ ID NO: 3 - DNA, 25 bp, Artificial Sequence
  Synthetic Oligonucleotide
ggtgcacctgactcctgtggagaag

SEQ ID NO: 4 - DNA, 25 bp, Artificial Sequence
  Synthetic Oligonucleotide
ggtgcacctgactcctgaggagaag

SEQ ID NO: 5 - DNA, 20 bp, Artificial Sequence
  Synthetic Oligonucleotide
gtgtctgtttgaggttgcta

SEQ ID NO: 6 - DNA, 15 bp, Artificial Sequence
  Synthetic Oligonucleotide
ctccttcaggagtca

SEQ ID NO: 7 - DNA, 15 bp, Artificial Sequence
  Synthetic Oligonucleotide
ctccaacaggagtca

SEQ ID NO: 8 - DNA, 54 bp, Artificial Sequence
  Synthetic Oligonucleotide
tagcaacctcaaacagacaccatggtgcatctgactcctgaggagaagtctgcc

SEQ ID NO: 9 - DNA, 54 bp, Artificial Sequence
  Synthetic Oligonucleotide
tagcaacctcaaacagacaccatggtgcatctgactcctgtggagaagtctgcc

SEQ ID NO: 10 - DNA, 20 bp, Artificial Sequence
  Synthetic Oligonucleotide
gaatgtgtcttggtctctgg